(19) United States
(12) Patent Application Publication
(10) Pub. No.: US 2024/0182945 A1
(43) Pub. Date: Jun. 6, 2024

(54) PRODUCTS FOR REGULATION OF EUKARYOTIC AND MICROBIAL CELLS GROWTH

(71) Applicants: Victor TETS, New York, NY (US); Georgy TETS, New York, NY (US)

(72) Inventors: Victor TETS, New York, NY (US); Georgy TETS, New York, NY (US)

(21) Appl. No.: 18/285,642

(22) PCT Filed: Apr. 5, 2022
(86) PCT No.: PCT/IB2022/053170

§ 371 Date: Oct. 4, 2023

Related U.S. Application Data
(60) Provisional application No. 63/170,838, filed on Apr. 5, 2021.

(51) Int. Cl. C12Q 1/04; C12N 1/20

(52) U.S. Cl. CPC C12Q 1/045; C12N 1/20

(57) ABSTRACT

The invention relates to methods for the flexible regulation of cellular growth of eukaryotic and prokaryotic cells. In particular embodiments. regulation of eucaryotic and microbial cells growth, occurs by the control of their interaction with the environmental factors, nutrient media, media additives, supplements.

### FIELD OF THE INVENTION

Provided herein are new automated systems, test systems, methods for preparation of test-systems for the control and regulation of eucaryotic and prokaryotic cells growth, regulation of microbial interaction with the physical, chemical, biological, environmental factors including antimicrobial agents and control of microbial diversity. Also provided are methods and products for the isolation of previously unculturable bacteria and fungi.

### BACKGROUND OF THE INVENTION

Today one of the critical issues for different areas of medicine, veterinary and biology is the ability to control and regulate eukaryotic and microbial cell growth and responses for the outer environment and to cellular diversity in vitro. The ability for the flexible regulation of cellular growth is the key for the use in a variety of process starting from laboratory practice, including the identification of previously unculturable microorganisms, selection of effective chemotherapeutic agents and in biomanufacturing. Such an in vitro modulation of eucaryotic and microbial cells growth requires accurate modulation of the cells' interaction with the environmental factors, nutrient conditions, media additives, supplements, ability to prepare highly individualized media with programmed composition in relation to the object of study, to control interaction of microorganisms and eucaryotic cells with physical, chemical, biological environmental factors, alter growth rate of different not-yet culturable bacteria, and/or fungi within mixed microbial communities and their interaction with the host factors.

### SUMMARY OF THE INVENTION

Various non-limiting aspects and embodiments of the invention are described below.

In some embodiments the method is used for cultivation of previously unculturable microorganisms; obtaining microorganisms with desired properties, directed selection, of microorganisms of interest with a non-limiting examples of Gram-positive, Gram-negative, rods, cocci, coccobacilli, vibrio, filamentous, spirochete.

In some embodiments when proposed method of cultivation enables the growth of previously unculturable bacteria with 16s rRNA sequence which is showing <97% identity grown on the medium as a pure or as a mixed bacterial culture

In one aspect, different bio samples contain bacteria and/or fungi and/or viruses.

In one aspect, different bio samples contain aerobic and/or anaerobic bacteria.

In some embodiments the PP and MM are bacteria and/or fungi.

In one aspect, different bio samples contain bacteria and/or fungi (including primary pathogens and/or microbial modulators) from the group consisting of bacteria, fungi (i.e. yeasts, molds,), protozoa with a non-limiting examples of bacteria and fungi with a non-limiting examples of Pseudomonadales, Aeromonadales, Legionellales, Pasteurellales, Vibrionales, Burkholderiales, Alphaproteobacteria, Spirochaetia, Lactobacillies, Bacillales, Enterobacterales, Ascomycota Basidiomycota Chytridiomycota Glomeromycota, Microsporidia, Myxomycota, Oomycota, Zygomycota, with a non-limiting examples of Aeromonas, Bacillus, Acinetobacter, Bartonella, Bordetella, Borrelia, Burkholderia, Brucella, Campylobacter, Chlamydia, Chlamydophila, Clostridium, Corynebacterium, Enterococcus, Escherichia, Haemophilus, Helicobacter, Klebsiella, Moraxella, Legionella, Leptospira, Listeria, Mycobacterium, Mycoplasma, Neisseria, Pseudomonas, Paenibacillus, Rickettsia, Salmonella, Shigella, Staphylococcus, Streptococcus, Treponema, Treponema, Ureaplasma Yersinia, Candida, Aspergillus, Mucor, Trichophyton, Blastomyces, Cryptococcus, Pneumocystis, Paracoccidioides, Histoplasma, Coccidioides, Talaromyces, Sporothrix. Emmonsia, Fusarium, Malassezia Microsporum Saccharomyces.

In some embodiments when microorganisms are analyzed pre- post- or together with PCR, transcriptome, metagenome sequencing, and other genetic-based analysis, biochemical, microbiological methods (i.e. staining, microscopy).

In some embodiments, antibiotics selected with test system is used to select antibiotics that can normalize patients state (with a non-limiting example of patients at critical state) and improving such a parameters as bacterial and/fungal load at the site of infection, bacterial and/fungal load at organs that are not the site of infection, bacterial and/fungal load in blood and other bodily fluids, pro-inflammatory markers, FEV1%, fever, edema, toxemia, survival rate, number and durations of hospitalization and/or antibiotic treatment, and/or decrease the ICU stay and/or decrease the post the initiation of antibiotic use and/or lead to a resolution of the infectious process and/or decrease the time required for wound healing and/or time of antibiotic administration and/or time of broad spectrum antibiotic administration within the first 96 h and/or from 4-28 days of selected antibiotic usage, alteration of the number of bacterial and fungal count from the site of infection and or other parts of the macroorganism.

In some embodiments antimicrobial agents selected are used for empirical therapy and/or adaptation of ongoing antimicrobial therapy

In some embodiments antimicrobial agents selected are used for prophylaxis

In some embodiments antimicrobial agents selected are used within the first 2.5 h of plating material

In some embodiments antimicrobial agents selected are used within the first 4 h of plating material

In some embodiments antimicrobial agents selected are used to overcome resistance, tolerance, intrinsic resistance, biofilm-associated tolerance, slow growth an persisters.

In one embodiment, persiters are selected from P. aeruginosa, Klebsiella, E. coli, S. aureus, Salmonella spp.,

In one embodiment, the method is used for the treatment of infections caused by bacteria and/or fungi that form mixed biofilms

In one embodiment, the method is used for the treatment of infections caused by multidrug resistant bacteria and to treat and/or prevent of recurrent infecti/ons

In some embodiments the proposed method is used for phage therapy

In some embodiments the antimicrobial agents and/or nucleases and/or other addictive are added directly to the biospecimen and/or media and the biospecimen is cultured at different magnetic conditions.

In one embodiment, the analysis of microbial growth and/or PP and/or MM is done by analyzing presence, metabolism, appearance or absence of microbial growth of mixed microbial cultures and/or analyzsis of morphology, size, color, biochemical, electrical and other characteristics of mixed bacterial communities or media where they grow.

In one embodiment, the analysis of microbial growth and/or PP and/or MM is done by analyzing presence, metabolism, appearance or absence of microbial growth to a predetermined threshold of growth.

In some embodiments PP and MM grow on the solid media

In some embodiments the diagnostic test system includes multi-well plate

In some embodiments the proposed method and devices create conditions in which the expression of antibiotic resistance genes of PP is similar to one at the site of the infection with a non-limiting example to be regulated by MM.

In some embodiments the nutrient media is additionally supplemented from the organic components from the site of the infection.

In some embodiments biosample is processed by adding a solvent (i.e. water, PBS, NaCl, nutrient media, biological material from the same subject), homogenization (i.e. vortex), filtration (i.e. include filtration step through 0.8, 1.0, 1.2-micrometer-pore-size filter to separate microorganisms from debris) is done.

In some embodiments the testing method uses biosamples stored at room temperature and/or at +4 and/or are frozen prior to processing.

In some embodiments from 1 to 10,000 different antimicrobial agents and combinations and/or permutation thereof are analyzed simultaneously against microorganisms of the same biosample.

In some embodiments when antibiotics are added according to PK/PD modeling and simulation in mammalian organism, modulate the adequacy of a given antibiotic regimen.

In some embodiments antimicrobial agents are added to the growth medium at concentrations representing various PK/PD parameters at the site of infection and/or systemic circulation and/or targeted organ

In some embodiments excipients and/or antimicrobial substances being investigated are introduced into the nutrient medium prior to cultivation of the microorganisms and/or at the same time and/or after and/or added to biosamples

In some embodiments wherein antibiotics efficacy against PP and/or MM is estimated within 1,2,3,4,5,6,7,8,9,10,11,12,13,14,15,16,17,18,19,20,21 22,23,24 or from 24-48 h, 48-120 h post plating.

In some embodiments nutrient media used for cultivation of bacteria and/or fungi including PP and/or MM contains Agar, Vitamin B12, Ascorbic Acid, Ammonium Nitrate, Ammonium Sulfate, Beef Extract, Alpha-Ketoglutarate, Beef Heart, Infusion, Bee Infusion, Biotin, Bitone H Plus Digest of Animal Tissue, Calcium Chloride, Cornstarch, L-Cysteine, Dextrose, Dipotassium Phosphate, D-Mannitol, Ferrous Sulfate, Folic Acid, Gelatin, Glucose, L-Cysteine HCl, Magnesium, Chloride, Magnesium Sulfate, Manganese Sulfate, Niacin, Nicotinamide, p-Aminobenzoic Acid, Pancreatic Digest of Casein, Pancreatic Digest of Gelatin, Pantone, Pantothenate, Peptic Digest of Animal Tissue, peptone, Potassium Sulfate, Proteose Peptone, Riboflavin, Sodium Chloride, Soluble Starch, Starch, Sucrose, Thiamine, Tris (Hydroxymethyl) Aminomethane, Tryptic Digest of Beef Heart, Tryptoneg, Yeast Extract, Zinc Sulfate, ACES Buffer, Potassium chloride, Thioglycollate, Mycological peptone (enzymatic digest of casein and animal tissues), Particles of cooked meat broth, Erythrocytes, Blood as well as constituents Source Amino-Nitrogen Peptone, protein hydrolysate, infusions and extracts Growth Factors Blood, serum, yeast extract or vitamins, NAD Energy Sources Sugar, alcohols and carbohydrates Buffer Salts Phosphates, acetates and citrates Mineral Salts and Metals Phosphate, sulfate, magnesium, calcium, iron Selective Agents Chemicals, antimicrobials and dyes Indicator Dyes Phenol red, neutral red Gelling agents Agar, gelatin, alginate, silica gel, water, Serum, FBS, Galactose.

In one embodiment test system following biosaple plating is cultivated at the temperature from 27 C to 42 C

In some embodiments when antibiotics selected against mixed culture of PP and MM combination test system allows to find effective narrow spectrum antibiotics that although being active solely against Gram-positive bacteria are found to be active against Gram-negative bacteria and/or when antibiotics solely active against Gram-negative bacteria being active against Gram-positive bacteria.

In one embodiment, automated system enables automatically to manufacture test system according to individual requests of the doctors, that may include the list of any antibiotics and their concentrations depending on the site of the infection and state of the patient.

In one embodiment, automated system has the reserve of the nutrient media, plates with different numbers of wells, supplements to the media and different antibiotics used in the clinical practice.

In one embodiment, automated system can in advance prepare test systems that are more frequently used and store them in appropriate temperature and humidity environment.

In some embodiments the analysis of the “appearance” or “progression” or absence of the signs of microbial growth when for the analysis the surface of the agar is done with (i) the certain fixed distance between the sample and the camera (ii) certain wavelength is used, (iii) certain diameter of the scattering spot and (iv) certain angle between the test system and the camera.

In one embodiment, the naked eye analysis is used for the analysis of the “appearance” or “progression” or absence of the signs of microbial growth and/or PP and/or MM

In one embodiment, the photo and/or video camera are used for the analysis of the “appearance” or “progression” or absence of the signs of microbial growth and/or PP and/or MM

In one embodiment, the photo and/or video camera are used with the exposition from and ISO from 50 to 12800 and the shutter speed from up to 30 seconds the analysis of the “appearance” or “progression” or absence of the signs of microbial growth and/or PP and/or MM

In one embodiment, special software is used to modify photo and/or video data used for the analysis of the “appearance” or “progression” or absence of the signs of microbial growth and/or PP and/or MM

In one embodiment, the analysis of the “appearance” or “progression” or absence of the signs of microbial growth and/or PP and/or MM is done with magnification from 0.1x to 1000x

In one embodiment, the analysis of the “appearance” or “progression” or absence of the signs of microbial growth and/or PP and/or MM is done using a fixed distance of between the sample and the detector (i.e. camera) with a non-limiting examples of 4-5 cm, 7-10 cm, 12-18 cm.

In one embodiment, the analysis of the “appearance” or “progression” or absence of the signs of microbial growth and/or PP and/or MM is done to avoid glares on the media or microbial colonies surfaces.

In one embodiment, the analysis of microbial growth and/or PP and/or MM in the same well is done over time with the image taken within any range from 1 to 300 minutes

In one embodiment, the analysis of microbial growth and/or PP and/or MM in multiple wells is done over time with the analysis taken within any range from 1 to 300 minutes and can be analyzed with naked eye and/or visualization software and/or AI.

In one embodiment, the analysis of microbial growth and/or PP and/or MM and or absence of microbial growth in the same and/or in multiple well is done constantly by video recording

In one embodiment, the analysis of microbial growth and/or PP and/or MM in the same well is done over time with the image talking within any range from 1 to 48 hours

In one embodiment, the analysis of microbial growth and/or PP and/or MM in multiple wells is done over time with the analysis talking within any range from 1 to 48 hours and can be analyzed with naked eye and/or visualization software and/or AI.

In one embodiment, the analysis of microbial growth and/or PP and/or MM in the same well is done over time with fluorescent dyes added to the medium or to the biosample and follow up cultivation on the medium supplemented with antimicrobial agents to visualize the presence, metabolism, appearance or absence of microbial growth by measuring levels of fluorescent intensity

In one embodiment, the analysis of microbial growth and/or PP and/or MM in the same well is done by measuring levels of fluorescent intensity identified with antibodies or isotopic labeling.

In one embodiment the study of antimicrobial activity of the compound added to the agar is done, based on the analysis of the “appearance” or “progression” or absence of the signs of microbial growth with naked eye comparing the growth between different time points and/or different antibiotics and/or antibiotics mix and/or absence of antibiotics

In one embodiment the study of antimicrobial activity of the compound added to the agar is done, based on the analysis of the “appearance” or “progression” or absence of the signs of microbial growth with optical instruments i.e. microscope with (1×, 1.5×, 2×-10×10×-40×, 40x-1000× magnification) with or without special dyes

In one embodiment the study of antimicrobial activity of the compound added to the agar is done, based on the analysis of the “appearance” or “progression” or absence of the signs of microbial growth with metabolomic analysis of the extracellular matrix substances

In one embodiment the study of antimicrobial activity of the compound added to the agar is done, based on the analysis of the “appearance” or “progression” or absence of the signs of microbial growth with photo comparing the growth between different time points and/or different antibiotics and/or antibiotics mix and/or absence of antibiotics

In one embodiment the study of antimicrobial activity of the compound added to the agar is done, based on the analysis of the “appearance” or “progression” or absence of the signs of microbial growth with video comparing the growth between different time points and/or different antibiotics and/or antibiotics mix and/or absence of antibiotics

In one embodiment, the test system is a microfluidic device.

In one embodiment the study of antimicrobial activity of the compound added to the agar is done, with specific dyes to detect microbial growth

In one embodiment the study of antimicrobial activity of the compound added to the agar is done, based on the analysis of the “appearance” or “progression” or absence of the signs of microbial growth with machine vision and/or AI algorithms and/or predictive algorithms

In one embodiment the study of antimicrobial activity of the compound added to the agar is done, based on the analysis of the “appearance” or “progression” or absence of the signs oof microbial growth based on the use of different readouts, with a non limiting readouts of 3D, dynamic, reflect light and sensor technology, use of the light with a specific waive length.

In one embodiment special tags with a non-limiting example of DNA tags to PP an/or MM are used to study microbial growth.

In one embodiment when the effect of antibiotics to inhibit bacterial growth is studied from 2 to 18 hours

In one embodiment, the automated devices are used to manufacture nutrient media and the cultivation of microorganisms, putting an additives to nutrient media, and methods of their use, which differ in that they allow the individual production of media with programmed compositions in relation to the object of study, control the interaction of microbes with environmental factors, change the intensity of growth of various including not yet cultivated bacteria and fungi, as part of mixed biofilms, to change the growth rate of microbes, to determine the effect of antibiotics on them, as well as devices and methods for recording the results of these actions.

In some embodiments the device for microbial growth provides an automated process for the preparation of a nutrient medium with certain parameters, plating biosamples on the medium, processing biosamples at certain temperature and/or magnetic field and/or CO2 content and reading data analysis for the particularities of microbial growth.

In one embodiment automated system is a special station capable based on a labeling on a plate to identify the baseline/raw data of a given test system, maintain conditions and temperatures necessary for microbial/cell growth, and periodically scan the probes to register the presence and/or absence and/or alteration of growth.

In some embodiment the automated system has a recording unit that allows to identify the presence of growth, analyze this data and formulate recommendations for the use of antimicrobial agents for the treatment of a given patient with the information being sent to the doctor.

In one embodiment when nutrient media used for cultivation of microbial growth (i.e. Primary Pathogen and/or Microorganisms Modulators) is additionally supplemented or has an internal and or external parts and/or coverings used to modify magnetic, electrical and/or electromagnetic filed with a non-limiting examples of using foil, metals, metals coverings, metal boxes, metal wells, special envelops, mu-metal, magnetic shielding, components having nickel-iron components, materials with ferromagnetic alloy, etc to alter magnetic and/or electromagnetic fields that affect microbial growth.

In some embodiments, when bacteria and/or fungi from the site of infection can be concentrated from the biosamples (with a non-limiting examples of blood, CSF, ascites) prior to plating on the test system with different methods with a non-limiting examples of centrifugation, and/or dissolved with added additional portion of biosample, nutrient media, buffer.

In some embodiments wherein for preparation of test-systems of different construction and/or cultivation of microorganisms with the use of nutrient media, media additives and supplements, methods of their usage, differs in that enables individually to prepare media with programmed composition in relation to the object of study and/or regulate, control interaction of microorganisms and eucaryotic cells with physical, chemical, biological environmental factors, and/or alter growth rate of different with a non-limiting examples not-yet culturable bacteria, fungi within mixed microbial communities, alter and/or enhance microbial growth and/or synthetic activity, and/or determine the effect of antimicrobial drugs on them to select effective and/or ineffective antimicrobial agents and/or combinations thereof including those with as narrow as possible spectrum of action and/or with the highest safety profile and/or cheapest and/or with certain route of administration active against (1) drug resistant microorganisms and/or (2) the existing persisters or persisters that can be formed in the course of antibiotic therapy of the infection and/or sporeforming bacteria, (3) recurrent infections done by (3) simultaneous analysis of combinational effect of antimicrobial agents on Primary Pathogen (PP) and Microorganisms Modulators (MM), as well as devices and methods of accounting for the results of these actions, wherein said sample comprising at least one of fluid, water, food, beverage, biofluid, tissue, microbial probes, a pharmaceutical preparation, a mammalian sourced tissue and liquids, air, soil, surface, swabs, and any combination thereof for the use in biopharmaceutical manufacturing, medicine, veterinary, agriculture, bioengineering, drug development and discovery, ecology, space exploration, space technology, persons with altered metabolic parameters that alters the pharmacokinetics and pharmacodynamics of drugs and/or children in particular

In some embodiments, wherein automatic or semiautomatic station/complex for the preparation of test-systems of different construction with individual programmed properties, set of antimicrobial compounds and different supplements in variable concentrations and combinations, marking all the details and properties of the final product and creating its electronic passport, preparing the testing material for sowing test systems, making the sowing of the testing material on the test system, ensuring the growth of microorganisms with different tolerance to oxygen under specified conditions, controlling the growth of microbes, taking into account the results of this growth and/or the results of the effectiveness of antibiotics, and entering the results into the database and/or sending them to the specified address/destination.

In some embodiments, wherein recording unit of automatic or semiautomatic station/complex that takes into account the results of the growth of microorganisms in the test system and/or the results of the effectiveness of antimicrobials, and adds the results to the database and/or sends them to the specified address/destination.

In some embodiments the device monitors the growth and/or takes into account the results of this growth and/or the results of the effectiveness of antibiotics at specified time intervals with a non-limited examples of digital photography, registration in the visible and/or ultraviolet and/or infrared spectra and/or computer-generated imagery with a non-limiting example of calculation the grayscale values and computing histograms, and/or photometry, and/or colorimetry and/or conductivity/resistance of medium of current flow.

In some embodiments wherein test system, in which sensors are integrated, allowing to monitor in various ways the state of individual cells, which changes depending on the presence and nature of the microorganisms' growth and metabolism

In some embodiments wherein the control of microbial growth on the surface of media is done from an angle of from 1 to 10 and/or 10 to 30 and/or 30-45 and/or 45-90 degrees to the detecting sensor.

In some embodiments, wherein the test system contains concentration gradients for each antimicrobial agents in one zone and/or in the form of separate isolated zones, reflecting their pharmacokinetics in certain tissues and organs from zero to the maximum amount.

In some embodiments wherein Bio adjustable Tetz incubators and/or CO2 incubators and/or incubator-warm rooms for the cultivation of bacteria and/or fungi and/or cells, that enable control and/or regulate geomagnetic field and/or electromagnetic exposure and/or alterations of geomagnetic activity and/or alterations of magnetic field of the earth.

In some embodiments wherein incubators, containers (with a non-limiting example of fermenters, tanks, bioreactors), envelopes, bags, labware, lab supplies, have a shell/part made of Mu metal that provides a given degree of protection and/or their complexes with dielectrics (for example, plastic or paper, having a combination of Copper Chambers with zink and asbestos).

In some embodiments wherein incubators, containers (with a non-limiting example of fermenters, tanks, bioreactors), envelopes, bags, labware, lab supplies, culture plates, have a shell/part made of foil and or their complexes with dielectrics.

In some embodiments wherein incubators, containers (with a non-limiting example of fermenters, tanks, bioreactors), envelopes, bags, labware, lab supplies, culture plates, have a shell/part made of Mu-metal and/or foil and/or their complexes with dielectrics.

In some embodiments wherein Mu-metal is used to alter and/or enhance microbial growth and/or synthetic activity and/or secretion and/or expression of genes with a non-limiting examples of natural and/or modified and/or engineered eucaryotic or procaryotic producers of molecules and/or proteins of interest, protein expression system, phage display, and those overexpressing recombinant proteins for the use in non-limiting examples of medicine, biotechnology, biomanufacturing, food industry.

In some embodiments wherein incubators, containers (with a non-limiting example of fermenters, tanks, bioreactors), envelopes, bags, labware, lab supplies, culture plates, having a shell/part made of Mu-metal and/or foil and/or their complexes with dielectrics for cultivation of cells used for the analysis of the environment, ecology with a non-limiting examples of geomagnetic alterations, magnetic field and/or waves, radiation.

In some embodiments supplements and additives for nutrient media and treatment of biosamples of claim 1, wherein they allow to regulate growth of different microorganisms including those within mixed microbial communities by the addition of ribavirin (from 0,1-1000 μg/mL), acyclovir (from 0,1-1000 μg/mL), lithium orotate from 0,1-1000 μg/mL), potassium orothate (from 0,1-1000 μg/mL), derivatives of 2-chloro-5-phenyl-5H-pyrimido[5′,4′:5,6]pyrano[2,3-d]pyrimidine-4-ol (from 0,1 -1000 ug/mL), nucleases (with a non-limiting examples of DNase (from 0,1 -1000 μg/mL) and/or RNase (from 0,1-1000 μg/mL), transcriptase and/or integrase inhibitors and/or protease inhibitors with a non-limiting examples of nevirapine, etravirine, lamivudine, tenofovir, abacavir, raltegravir (from 0,1-1000 μg/mL), that can be used to modulate microbial growth and/or synthetic activity and/or secretion and/or expression of genes, the ratio of growth of different microorganisms including affecting the proportion of Firmicutes, Gracilicutes Mollicutes, acid fast bacteria, yeasts, molds and eukaryotic cells cultures.

In some embodiments nutrient media is used for the accelerated growth of fungi (dermatophytes, yeasts, molds) with a non-limiting examples of Candida, Aspergillus, Mucor, Trichophyton, Blastomyces, Cryptococcus, Pneumocystis, Paracoccidioides, Histoplasma, Coccidioides, Talaromyces, Sporothrix. Emmonsia, Fusarium, Malassezia Microsporum Saccharomyces Saprolegnia Erysiphe, Clavicens, Cladosporium. Bipolaris, Shoem, Helmintosporium, Alternaria Penicillium Cladosporium, Alternaria, Epicoccum, Aureobasidium, Absidia Chrysosporium Geotrichum Risopus Eurotium, including combined growth of different fungi and/or bacteria within mixed microbial communities, that can be found in the outer environments, soil, water, books, art objects, objects that interact with humans, animals plants, fungi that cause mammalian, plant diseases comprising of: potato decoction from 0.1 to 500 ml; corn flour tincture from 0.1 to 100 mL; oat flour from 10 to 350 mL; Heart-brain broth from 0,001 to 100,0 g; potato-carrot decoction from 10 to 350 mL; Dextrose/Glucose 40 g; Peptone 10 g; Sucrose from 0,001 to 100,0 g; Cellobiose, from 0,001 to 100,0 g; Yeast extract from 0,001 to 20,0 g; Maltose from 0,001 to 100,0 g; NaNO3 from 0,001 to 10,0 g; K2HPO4 from 0,001 to 10,0 g; MgSO4 from 0,001-10,0 g; KCl from 0,001 to 10,0 g; FeSO4 from 0,001 to 40,0 g; Zn SO4 from 0,001 to 5,0 g; MnCl2 from 0,001to 5,0 g, Twin 80 from 0,001 to 10 mL, Thiamine from 0,001 to 5,0 mg Biotin from 0,001 to 4,0 mg; brain-heart broth 0,001-100,0 g, Agar from 0 to 100 g, orotate derivatives from 0,001 to 500 g; erythrocytes from 0 to 100 mL, antibiotics to inhibit bacterial growth (such as chloramphenicol, chloramphenicol, cephalosporins, tetracycline).

In some embodiments, wherein the test system is used for the express growth of fungi (dermatophytes, yeasts, molds) with a non-limiting examples of Candida, Aspergillus, Mucor, Trichophyton, Blastomyces, Cryptococcus, Pneumocystis, Paracoccidioides, Histoplasma, Coccidioides, Talaromyces, Sporothrix. Emmonsia, Fusarium, Malassezia Microsporum Saccharomyces Saprolegnia Erysiphe, Clavicens, Cladosporium. Bipolaris, Shoem, Helmintosporium, Alternaria Penicillium Cladosporium, Alternaria, Epicoccum, Aureobasidium, Absidia Chrysosporium Geotrichum Risopus Eurotium.

In some embodiments wherein for the express selection of antibiotics effective against fungi, wherein the antifungal agents are selected from the non-limiting examples of azole derivatives (ketoconazole, fluconazole, isavuconazole, itraconazole, posaconazole, and voriconazole), Echinocandins (anidulafungin, caspofungin, Aminocandin, micafungin), allylamine (terbinafine, Naftin, Tolnaftate), polyene (nystatin, amphotericin B) Flucytosine, Ibrexafungerp, antiseptics, disinfectants.

In some embodiments wherein test system enables the simultaneous growing the maximum number and/or diversity of unrelated microorganisms present at the site of infection, allowing to register the early growth of microorganisms by various methods and to determine the efficiency of the use of antimicrobial agents added to the medium due to their action on Microbial Modulator(s) which control at the site of infection the properties of Primary Pathogen(s), by cultivating biosamples and/or bacteria and/or fungi on the medium with a non-limiting example of: Bile Salt Agar, Thiosulphate Citrate Bile Salts-Sucrose Agar, Bile Esculin Agar, Blood Agar, Chockolate agar, Charcoal Blood Agar, Brain Heart Infusion Broth, Cycloserine Fructose Agar, Cycloserine Egg-Yolk Agar, Egg Saline Medium, Alkaline Egg Medium, Blood-Digest Agar and Broth, Fletcher's Agar, Heated Blood Agar/Chocolate Agar, MacConkey Agar, Mannitol Salt Agar, Hiss's Serum Water Medium, Loeffler Serum Mueller-Hinton Agar, Nutrient Agar, Cooked meat broth, Non-Nutrient Agar Peptone Water, Pike's Media, Anaerobically Sterilized media, Robertson Cooked Meat Broth, Semisolid Agar, Campylobacter Medium, Gram-Negative Broth, Trypticase Soy Broth, Thioglycollate Broth, Trypticase Soy Broth, minimal media, corn meal agar, potato dextrose agar, V-8 juice agar, and dung agar yeast extract, malt extract agar, (Salmonella-Shigella) Agar, Hektoen enteric agar , Listeria comprises a composition of Listeria Broth, DMEM, Fetal Calf Serum, Nalidixic acid, FBS , Tetrathionate Broth, Sabouraud's agar, charcoal yeast extract agar, Mannitol salt agar, LB broth, LB agar, Columbia broth, Columbia agar, Pepted Meat agar, MPB, BcS-LM growth medium, Brucella agar Cornmeal Agar, Water Agar, Emerson's YpSs agar Antibiotic Agar, Acidified Cornmeal Agar, Potato Carrot Agar, Malt Agar, Malt Extract Agar, Potato Dextrose Agar, and a combinations thereof.

In some embodiments wherein to obtain pure culture of cultivated and not-yet-cultivated microorgnaisms using a test system of claims 1 and 2 in which microorganisms are subcultured from zones and/or wells containing and/or not containing antimicrobial agents.

In some embodiments wherein the analysis of the signs of appearance and/or progression or absence of the signs of microbial growth (i.e. Primary Pathogen and/or Microorganisms Modulators) is done by visual examination (i.e. naked eye, microscope), or image detection with a non-limiting examples of photography, video, computer-generated imagery, photometry, colorimetry with a non-limiting example of calculation the grayscale values and computing histograms, with or without of automated program and/or AI algorithm and/or software; Image Recognition and Image Processing methods, spectrophotometry, scanners, lasers, with a non-limiting examples when the analysis of the surface of the media is done with (i) the certain fixed distance between the sample and the camera (ii) certain wavelength is used, (iii) certain angle between the test system and the camera (with a non-limiting example of from 1 to 10 and/or 10 to 30 and/or 30-45 and/or 45-90 degrees angle to the detecting sensor); is done by comparison of the photo images of the same wells supplemented with antimicrobial agent of interest within a certain time period or any combinations thereof and/or comparison with the growth in other wells and/or comparison with a predetermined threshold of growth.

In some embodiments wherein the results obtained by the test system allow to get data on the effectiveness of antimicrobial agents as soon as possible based on pairwise comparison of microbial growth of the same wells over the different time periods when antibiotic efficacy is evaluated by monitoring of the signs of appearance and/or progression or absence of the signs of microbial growth (i.e. Primary Pathogen and/or Microorganisms Modulators) within a certain time period or any combinations within a non-limiting examples of below listed time periods from: 0 to 1 hour, 0 to 2 h, 1 h to 2 h, 0 h to 3 h, 1 h to 3 h, 2 h to 3 h, 0 h to 4 h, 1 h to 4h, 2 h to 4 h, 0 to 5 h, from 1 h to 5 h, 2 h to 5 h, 3 h to 5 h, 0 h to 6 h, 1 h to 6 h, 2 h to 6 h, 3 h to 6 h, 4 h to 6 h, 9 h to 8 h, 1 h to 8 h, 2 h to 8 h, 3 h to 8 h, 4 h to 8 h, 0 h to 9 h, 1 h to 9 h, 2 h to 9 h, 3 h to 9 h, 4 h to 9 h, 5 h to 9 h, 0 h to 12 h, 1 h to 12 h, 2 h to 12 h, 4 h to 12 h, 8 h to 12 h, 0 h to 18 h, 1 h to 18 h, 2 h to 18 h, 4 h to 18 h, 6 h to 18h, 0 h to 24 h, 1h to 24 h, 2 h to 24 h, 4 h to 24 h, 12 h to 36 h, 24 h to 36 h.

In some embodiments wherein antibiotic efficacy is evaluated by monitoring of the signs of appearance and/or progression or absence of the signs of microbial growth (i.e. Primary Pathogen and/or Microorganisms Modulators) on the agar of the wells containing antibiotic of interest at different timepoints including fixed time of 2, 2.5, 3, 3.5, 4, 4.5, 5, 5.5, 6, 6.5, 7, 7.5, 8 hours of growth.

In some embodiments wherein biosample is plated to the test system with antimicrobial agents added to the medium taken at concentrations (less than maximum concentration at the site of infection) that corresponds to the mean concentration of the antimicrobial agents achievable at the site of infection and/or systemic circulation at different time points after this antimicrobial agents administration with a non-limiting examples of: antimicrobial concentration from 0 to 1 hour (C0-1h), C1-2h, C0-2h, C1-2h, C0-3 h, C1-3h, C2-3h, C0-4h, C1-4h, C2-4 h, C3-4h, C0-5h, C1-5h, C2-5h, C3-5h, C4-5h, C0-6h, C1-6h, C2-6h, C3-6h, C4-6h, C5-6h, C0-7h, C1-7 h, C2-7 h, C3-7h, C4-7h, C5-7h, C0-12h, C2-12h, C4-12h, C6-12h, C8-12h, C0-24h, C6-24h, C12-24h.

In some embodiments wherein the tets system contains medium supplemented with antimicrobial agent(s) which concentration is selected from the values that can be reached at the site of infection for the time sufficient to kill and/or inhibit microbial growth (with a non-limiting examples of time within 30 minutes, 2 hours, 3 hours).

In some embodiments according to claims 1-23, wherein antibiotics efficacy models are assigned with different probabilities

In some embodiments the test system allows as quickly as possible (with a non-limiting example from 2 to 6 h) select antibiotic that is selectively active against certain bacteria and/or fungi within microbial mix

In some embodiments wherein antibiotic concentrations added to the test system are selected based on particularities of pharmacokinetics that depends on the rout of its administration to the individual with a non-limited examples topically, enterically, orally, parenterally, inhaled, intranasal, rectal, vaginal.

In some embodiments wherein antibiotics in the test system are selected to treat bacterial and/or fungal infections with a non-limiting examples of Ear infections, Sinus infections, Cough or bronchitis, Sore throat, pulmonary infections (pneumonia, cystic fibrosis, chronic obstructive pulmonary disease, tuberculosis, mycobacterium, histoplasmosis, blastomycosis bronchiectasis, abscesses, empyema) skin and soft tissue infections (diabetic wound infection, burns, wounds, bites, Impetigo, Cellulitis/Erysipelas, Folliculitis, Skin Abscess, Furuncle, Carbuncle, Necrotizing Soft Tissue Infections), gynecological infections, Maternal infections, ophthalmic infections, oropharyngeal infections, infections of gastrointestinal tract (poisoning, IBD, Inflammatory bowel disease), meningitis, sepsis, fungaemia, systemic mycosis, onychomycosis, urinary tract infections, sexually transmitted diseases, vulvovaginal candidiasis, in individuals with normal or compromised immune response, infections caused by Burkholderia spp, including those in children and patients undergoing lung transplantation, infections associated with persister formation and/or preventing their formation and/or caused by sporeforming microorganisms, and/or recurrent infections

In some embodiments wherein culture media and antibiotics are used (taken in different concentrations) in the test system that allow the isolation of previously unculturable microorganism (PP and/or MM) from mixed communities.

In some embodiments wherein antimicrobial effect of drug candidates and/or drugs is evaluated against mixed microbial communities (with a non-limiting examples of microbial communities within biosamples) for (1) comparative analysis with the activity of other drugs (2) select patient population for the clinical trials (3) evaluating the effectiveness of action on humans and animals.

In some embodiments wherein the algorithm of antibiotic selection is based on the evaluation of the highest probability to be effective is determined by which “X” value in “C 1/x max” equation is the highest.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the plan of the automatic or semiautomatic station/complex for the preparation of test-systems.

FIG. 2 shows the plan of the registration unit of automatic or semiautomatic station/complex for the preparation of test-systems.

FIG. 3 shows the analysis of presence of growth following naked eye examination with the follow up monitoring for 24 h using Method (Algorithm) #1 or Method (Algorithm) #2* MM—mixed medium (Columbia agar+Pepted Meat agar); LB—Luria Broth agar; PMA—Pepted Meat agar; CA—Columbia agar; SA—Schaedler agar

FIG. 4 shows the analysis of presence of growth following microscopy examination with the follow up monitoring for 24 h using Method (Algorithm) #1 or Method (Algorithm) #2* MM-mixed medium (Columbia agar+Pepted Meat agar); LB—Luria Broth agar; PMA—Pepted Meat agar; CA—Columbia agar; SA—Schaedler agar. * Combined data for the whole set of antibiotics used.

FIG. 5 shows the analysis of absence of growth following naked eye examination with the follow up monitoring for 24 h using Method (Algorithm) #3*

FIG. 6 shows the analysis of absence of growth following microscopy examination with the follow up monitoring for 24 h using Method (Algorithm) #3*MM—mixed medium (Columbia agar+Pepted Meat agar); LB—Luria Broth agar; PMA—Pepted Meat agar; CA—Columbia agar; SA—Schaedler agar *Combined data for the whole set of antibiotics.

FIG. 7 shows the analysis of presence of growth following naked eye examination with the follow up monitoring for 24 h using Method (Algorithm) #1 or Method (Algorithm) #2* *Combined data for the whole set of antibiotics used.

FIG. 8 shows an example of greyscale histogram of a single probe used in the study

FIG. 9 shows the effect of different magnetic and/or electromagnetic fields on diversity of microbial growth.

FIG. 10 shows the analysis of absence of growth following naked eye examination with the follow up monitoring for 24 h. CC—control; D—DNase; R—Rnase; D+R—DNase+RNase.

FIG. 11 shows bacterial diversity under the growth at normal magnetic field, altered magnetic field (foil), altered magnetic field (Mu-shield material) in the presence of absence of antibiotics.

FIG. 12 shows the use of foil to increase cell number (×10 magnification)

FIG. 13 shows the effect of different novel incubators on the growth of microorganisms

FIG. 14 shows the use Mu-metal containing devices to monitor environmental conditions.

FIG. 15 shows the analysis of presence of growth following naked eye examination with the follow up monitoring up to 24 h

FIG. 16 shows the use of u-metal and different agar settings to regulate bacterial growth

FIG. 17 shows the use of u-metal and different agar settings to regulate bacterial memory

FIG. 18 shows the use of u-metal test systems to monitor health state

FIG. 19 shows the use of light on microbial growth in normal and altered geomagnetic conditions

### DETAILED DESCRIPTION OF THE INVENTION

Here we for the first time unexpectedly identified a novel method of eukaryotic and prokaryotic cells control growth; development of an automated diagnostic methods and test systems including those enabling selection of: effective narrow-spectrum antibiotics, antibiotics effective against antibiotic resistant bacteria including those against which this antibiotic is not effective when they cultured as a manobacteria; prevent persisters formation; antibiotics effective against persisters that are already present at the site of infection or the organism, for organisms with individual particularities of antimicrobial agents' metabolism, by modeling growth of microorganisms identical to those in vivo, and modelling and/or regulation of such a process by changing cell diversity. The implementation of the method is further explained by means of the examples provided below.

### Definitions

Primary Pathogen—Microorganism that in specific conditions can be a disease causative agent. At the site of infection there can be one or multiple Primary Pathogens. Primary Pathogen are selected from the group consisting of bacteria, fungi (i.e. yeasts, molds,), protozoa with a non-limiting examples of bacteria and fungi with a non-limiting examples of Pseudomonadales, Aeromonadales, Legionellales, Pasteurellales, Vibrionales, Burkholderiales, Alphaproteobacteria, Spirochaetia, Lactobacillies, Bacillales, Enterobacterales, Ascomycota Basidiomycota Chytridiomycota Glomeromycota, Microsporidia, Myxomycota, Oomycota, Zygomycota, with a non-limiting examples of Aeromonas, Bacillus, Acinetobacter, Bartonella, Bordetella, Borrelia, Burkholderia, Brucella, Campylobacter, Chlamydia, Chlamydophila, Clostridium, Corynebacterium, Enterococcus, Escherichia, Haemophilus, Helicobacter, Klebsiella, Moraxella, Legionella, Leptospira, Listeria, Mycobacterium, Mycoplasma, Neisseria, Pseudomonas, Paenibacillus, Rickettsia, Salmonella, Shigella, Staphylococcus, Streptococcus, Treponema, Treponema, Ureaplasma Vibrio, Yersinia, Candida, Aspergillus, Mucor, Trichophyton, Blastomyces, Cryptococcus, Pneumocystis, Paracoccidioides, Histoplasma, Coccidioides, Talaromyces, Sporothrix. Emmonsia, Fusarium, Malassezia Microsporum Saccharomyces Saprolegnia Erysiphe, Clavicens, Cladosporium. Bipolaris, Shoem, Helmintosporium, Alternaria Penicillium Cladosporium, Alternaria, Epicoccum, Aureobasidium, Absidia Chrysosporium Geotrichum Risopus Eurotium

Microbial Modulator—Microorganism that in specific conditions can directly or indirectly modulate Primary Pathogens, increasing or decreasing sensitivity of Primary Pathogens to antibiotics including changing expression of antibiotic resistance genes.

Antibiotic efficacy—when antibiotics efficacy against the “principal pathogen” are selected from those antibiotics that directly or indirectly affect (with a non-limiting option kill, eliminate, decrease viable counts, inhibit growth) of Primary Pathogen and/or Microorganisms Modulators

Appearance and/or progression of signs of microbial growth include formation of film, microcolony, colony, lawn, biofilm, change in the color, change in the color of dyes, electrical and/or magnetic parameters that reflect the presence of cell growth and/or metabolism, change of fluorescence, etc.

Nutrient media—culture media suitable for microbial growth and/or used for cultivation of Primary Pathogen and/or Microorganisms Modulators and is a liquid media, semisolid media, dense media

Antimicrobial agents—are antimicrobial agents and/or combinations of thereof and/or permutation thereof with a non-limiting examples of Aminoglycosides, Annamycin, Penicillins, Macrolides, Cephalosporins, Chloramphenicol, Glycopeptides, Fluoroquinolones, Beta-lactams with increased activity, Tetracyclines, Quinolones, Sulfosamides, Streptogramins, Trimethoprim sulfamethoxazole, Urinary anti-infective, lipopeptides, oxazolidinones, annamycin, nitrofurantoin, nitroimidazole, Lincosamides, azoles, echinocandin, nitroimidazole, polyene antibiotics, triterpenoids, peptide antimicrobial agents, bacteriophages, as well as antiseptics and disinfectants (i.e. alcohols, aldehydes, anilids, biguanides, phenols, diamidines, halogen releasing agents, metal derivatives, peroxygens, quaternary ammonium compounds, vapour phase)

Bio adjustable Tetz incubators—incubators and/or CO2 incubators and/or [symbol] incubator-warm rooms for the cultivation of bacteria and/or fungi and/or cell cultures, that enable control and/or regulate geomagnetic field and/or electromagnetic exposure and/or alterations of geomagnetic activity and/or alterations of magnetic field of the earth.

Bio adjustable Tetz envelope/containers—that enable to control and/or regulate influence of geomagnetic field, radiation, electromagnetic emissions, wavering of geomagnetic activity, alterations of magnetic field of the earth an other.

Biosamples comprising at least one of a non-limiting examples of mammalian sourced tissue and fluid e.g. saliva, swabs, sputum, broncho-alveolar lavage, pus, synovial fluid, biofliuids (e.g. blood serum, fetal blood serum, plasma, cerebrospinal fluid), breast milk, urine, wound and/or burn material, surgical material, digestive tract tissue, skin, epithelial tissue, connective tissue, muscular tissue, adipose tissue, areolar tissue, somatic tissue, neuronal tissue, bone tissue, cartilage tissue, lymphatic tissue, muscular tissue, fibrous tissue, urinary tract tissue, lymphatic tissue, liver tissue, and any combination thereof that can additionally be processed by a non-limiting examples of adding a solvent, homogenization, filtration

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

Revers transcriptase inhibitors (Nevirapine, Penciclovir, Tenofovir disoproxil, Zidovudine, Foscarnet, Efavirenz, Stavudine, Delavirdine, Lamivudine, Adefovir dipivoxil Etravirine Abacavir)

Integrase/recombinase inhibitors (raltegravir, Dolutegravir, Bictegravir, Cabotegravir

2,8-dichloro-5-(4-nitrophenyl)-5,9-dihydro-4H-pyrimido[5′,4′:5,6]pyrano[2,3-d]pyrimidine-4,6(1H)-dione (VTL)

Proteases inhibitors (Asunaprevir, Boceprevir, Grazoprevir, Glecaprevir, Paritaprevir, Simeprevir, Telaprevi, Amprenavir, Atazanavir, Darunavir, Fosamprenavir, Indinavir, Lopinavir, Nelfinavir, Ritonavir, Saquinavir, Tipranavir)

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Automated Test-System for Preparation of Test Systems, Biosamples Processing, Test Systems Processing, Data Registration and Analysis

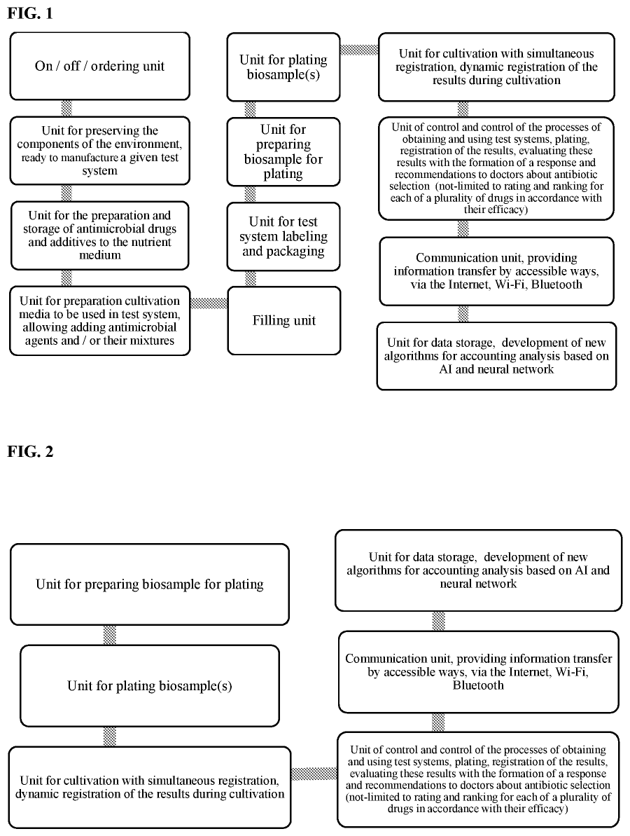

An example of the device that is an automatic or semiautomatic station/complex for the preparation of test-systems with individual programmed properties is presented in FIG. 1.

An example of the registration unit is presented in FIG. 2.

FIGS. 1 and 2 show possible structure of automated test-system for preparation of test systems, biosample processing, test system processing, data registration and analysis.

### Example 2. Determination of the Optimal Visualizing Angle for Data Analysis

We studied, could the visualizing angle affect the speed and precision of data analysis.

We plated endotracheal aspirate and sputum samples from patient with cystic fibrosis. Samples were dissolved with PBS and plated on the wells of multi-well plates filled with the mixed medium (Columbia agar+Pepted Meat agar+5% erythrocytes) with the growth area of 1.9, 0.95, 0.32 cm2. Analysis of the presence of microloconies or bacterial lawn were analyzed, when plates were visualized with the naked eye under different angles. Bacterial lawn was suggested as the appearance of bacterial colonies when all the individual colonies on a Petri dish agar plate merge to form a field or mat of bacteria. Data are presented in Tables 1 and 2.

TABLE 1
Appearance of microcolonies (hours)
depending on visualization angle

| | Appearance of microcolonies (hours) | | | | | | | | |
|---|---|---|---|---|---|---|---|---|---|
| | depending on visualization angle | | | | | | | | |
| Biosample | 0- | 11- | 21- | 31- | 41- | 51- | 61- | 71- | 81- |
| # | 10° | 20° | 30° | 40° | 50° | 60° | 70° | 80° | 90° |
| 1 | 3.5 | 3.5 | 3.5 | 3.5 | 3.5 | 3.5 | 3.5 | 3.5 | 4.5 |
| 2 | 3.5 | 3.5 | 3.5 | 3.5 | 3.5 | 4 | 3.5 | 4 | 6 |
| 3 | 3.5 | 3.5 | 3.5 | 3.5 | 3.5 | 3.5 | 3.5 | 3.5 | 4.5 |

TABLE 2
Appearance of lawn (hours) depending on visualization angle

| | Appearance of lawn (hours) | | | | | | | | |
|---|---|---|---|---|---|---|---|---|---|
| | depending on visualization angle | | | | | | | | |
| Biosample | 0- | 11- | 21- | 31- | 41- | 51- | 61- | 71- | 81- |
| # | 10° | 20° | 30° | 40° | 50° | 60° | 70° | 80° | 90° |
| 1 | 12 | 9 | 10 | 9 | 9 | 10 | 11 | 11 | 12 |
| 2 | 12 | 12 | 8 | 8 | 12 | 14 | 14 | 15 | 18 |
| 3 | 12 | 10 | 8 | 8 | 8 | 10 | 12 | 12 | 12 |

These results clearly show that the “appearance of microcolonies” as a sign of microbial growth, enables quicker evaluation of antibiotic efficacy, compared with “lawn” at any visualizing angle. Surprisingly, we found that the change of visualizing angle enables to visualizes microbial growth significantly faster.

### Example 3. Effect of Data Analysis Algorithm on Speed and Accuracy of Antibiotic Selection

Endotracheal aspirate (n=3), sputum (n=3), BAL (n=2), wound swab (n=3) from patients with ventilator associated pneumonia were resuspended in PBS and 0.15 μL were plated on 24 well plate test system filled with Mixed medium (Columbia agar +Pepted Meat agar) (MM), Luria Broth (LB) agar, Pepted Meat agar (PMA), Columbia agar (CA), or Schaedler agar (SA).

22 wells were filled with media supplemented with antibiotics (taken at mean concentration of each antibiotic achieved in lungs from 0 to 4 hours post drug administration): Ampicillin, Ceftriaxone, Ceftazidime, Cefepime, Piperacillin-tazobactam, Vancomycin, Clindamycin, Ciprofloxacin, Imipenem, Levofloxacin, Tobramycin, Amikacin, Linezolid, Tobramycin, piperacillin-tazobactam, ceftazidime+Tobramycin, piperacillin-tazobactam+Tobramycin. Two wells were antibiotic free.

Biosamples were plated to the medium with a sterile swab with a slight pressure to the agar and making circular movements. Plates was incubated at 37 C and the analysis of the of microbial growth was monitored every 15 minutes from 9 h to 5 h, then hourly from 5 h to 8 h and then at 18 and 24 h post plating with a naked eye or with an AmScope microscope on 20× magnification with 12 mp camera.

Three methods (algorithms) how to analyze bacterial growth were used.

Method (Algorithm) #1. Lab technician, analyzed the appearance of bacterial growth by comparing the signs of bacterial growth in each well supplemented with antibiotic(s) at certain timepoint, with the signs of bacterial growth in the same well at previous time point(s).

Method (Algorithm)#2. Lab technician analyzed the appearance of bacterial growth by comparing the signs of bacterial growth in each well supplemented with antibiotic(s) with bacterial growth in antibiotic-free control

Method (Algorithm) #3. Lab technician analyzed the absence of bacterial growth in the wells in fixed time period of 3.0 h, 3.5 h; 4 h and 24 h. The results are shown in Tables 3 and 4.

TABLE 3
Analysis of the signs of bacterial growth following
naked eye examination in the samples that
were resistant to a certain antibiotic.

| | Mean hour at which bacterial growth became visible | | | | | | | | | |
|---|---|---|---|---|---|---|---|---|---|---|
| | on the wells supplemented with a certain antibiotic | | | | | | | | | |
| | Method (Algorithm) #1 | | | | | Method (Algorithm) #2 | | | | |
| Antibiotic | MM | LB | PMA | CA | SA | MM | LB | PMA | CA | SA |
| No Antibiotic | 3 | 3.5 | 4.5 | 4 | 4.5 | 4 | 5.5 | 5.5 | 4.5 | 4.5 |
| Ampicillin | 3.5 | 4 | 3.5 | 3.5 | 4 | 4.5 | 5 | 5 | 5 | 5 |
| Ceftriaxone | 3 | 4 | 4 | 4.5 | 4 | 5 | 6 | 5 | 5 | 5 |
| Ceftazidime | 3.5 | 4.5 | 4.5 | 3.5 | 4 | 4.5 | 4.5 | 4.5 | 4.5 | 4.5 |
| Cefepime | 3 | 3 | 3 | 4 | 3.5 | 4.5 | 5.5 | 6 | 5.5 | 5 |
| Piperacillin- | 3.5 | 4 | 3.5 | 3.5 | 4 | 4 | 5 | 4 | 5.5 | 5.5 |
| tazobactam | | | | | | | | | | |
| Meropenem | 3.5 | 3.5 | 4.5 | 4 | 4 | 4.5 | 4.5 | 4.5 | 4.5 | 5.5 |
| Vancomycin | 3.5 | 3.5 | 4 | 3.5 | 4.5 | 4 | 5 | 6 | 5 | 5 |
| Clindamycin | 3 | 4.5 | 4 | 5 | 4 | 4.5 | 4.5 | 4.5 | 4 | 4.5 |
| Ciprofloxacin | 3.5 | 4 | 3.5 | 3.5 | 4.5 | 4.5 | 5.5 | 4.5 | 4.5 | 5 |
| Imipenem | 4 | 4 | 4 | 4 | 4 | 4 | 5 | 4.5 | 5 | 4.5 |
| Levofloxacin | 3 | 5 | 5 | 3.5 | 4.5 | 4.5 | 6 | 5 | 5 | 6 |
| Tobramycin | 3 | 4 | 3.5 | 3.5 | 3 | 4.5 | 5.5 | 5 | 6 | 5.5 |
| Amikacin | 3.5 | 4.5 | 3.5 | 5 | 4 | 4 | 5 | 4.5 | 5 | 5 |
| Linezolid | 3 | 3.5 | 3.5 | 5 | 3.5 | 3.5 | 6.5 | 5.5 | 4.5 | 4.5 |
| Tobramycin | 3 | 4 | 3.5 | 3.5 | 4 | 4 | 4 | 5 | 4 | 5 |
| piperacillin- | 3 | 5 | 3.5 | 4.5 | 4.5 | 4 | 5.5 | 6 | 5 | 5.5 |
| tazobactam + | | | | | | | | | | |
| Cefepime | | | | | | | | | | |
| ceftazidime + | 3 | 4 | 4 | 4 | 4.5 | 4 | 5.5 | 6 | 6 | 5.5 |
| Tobramycin | | | | | | | | | | |
| piperacillin- | 3.5 | 4 | 3.5 | 3.5 | 4 | 4.5 | 5 | 5 | 5 | 4.5 |
| tazobactam + | | | | | | | | | | |
| Tobramycin | | | | | | | | | | |
| Mean | 3.3 | 4.0 | 3.9 | 4.0 | 4.1 | 4.3 | 5.2 | 5.1 | 4.9 | 5.0 |

MM—mixed medium (Columbia agar + Pepted Meat agar);
LB—Luria Broth agar;
PMA—Pepted Meat agar;
CA—Columbia agar;
SA—Schaedler agar

TABLE 4
Analysis of bacterial growth following microscopy examination

| | Mean hour at which bacterial growth became visible | | | | | | | | | |
|---|---|---|---|---|---|---|---|---|---|---|
| | on the wells supplemented with a certain antibiotic | | | | | | | | | |
| | Method (Algorithm) #1 | | | | | Method (Algorithm) #2 | | | | |
| Antibiotic | MM | LB | PMA | CA | SA | MM | LB | PMA | CA | SA |
| No Antibiotic | 3 | 3 | 4 | 3.5 | 4 | 4.5 | 4.5 | 4.5 | 4.5 | 4.5 |
| Ampicillin | 3 | 3 | 3.5 | 3 | 4 | 4 | 5 | 5 | 5 | 5 |
| Ceftriaxone | 3 | 3.5 | 3.5 | 4.5 | 3 | 4.5 | 5 | 4.5 | 4.5 | 5 |
| Ceftazidime | 3.5 | 4 | 4 | 3.5 | 3.5 | 4.5 | 4.5 | 4.5 | 4.5 | 4.5 |
| Cefepime | 3 | 3 | 3 | 3.5 | 3 | 4.5 | 5 | 5 | 4.5 | 5 |
| Piperacillin- | 3.5 | 4 | 3.5 | 3.5 | 3.5 | 4 | 4 | 4 | 3.5 | 3.5 |
| tazobactam | | | | | | | | | | |
| Meropenem | 3.5 | 3.5 | 4 | 3.5 | 4 | 4.5 | 4.5 | 4.5 | 4.5 | 4.5 |
| Vancomycin | 3.5 | 3.5 | 3.5 | 3.5 | 4 | 4 | 4 | 4 | 4 | 4 |
| Clindamycin | 3 | 3 | 3.5 | 4.5 | 4 | 4.5 | 4.5 | 4.5 | 4.0 | 4.5 |
| Ciprofloxacin | 3.5 | 4 | 3 | 3.5 | 4 | 4.5 | 4.5 | 4.5 | 4.5 | 4.0 |
| Imipenem | 4 | 4 | 4 | 3 | 4 | 4 | 5 | 4.5 | 3.5 | 4.5 |
| Levofloxacin | 3 | 4 | 4 | 3.5 | 3.5 | 4.5 | 5.5 | 4.5 | 4.5 | 5.5 |
| Tobramycin | 3 | 3 | 3.5 | 3 | 3 | 4.5 | 5.5 | 5 | 5.5 | 5.5 |
| Amikacin | 3 | 4 | 3.5 | 4.5 | 3.5 | 4 | 5 | 4.5 | 5 | 5 |
| Linezolid | 3 | 3 | 3 | 4 | 3.5 | 3.5 | 4.5 | 4.5 | 4.5 | 4.5 |
| Tobramycin | 3 | 3.5 | 3.5 | 3 | 3 | 4 | 3.5 | 3.5 | 4 | 4 |
| piperacillin- | 3 | 4 | 3 | 4 | 4 | 4 | 4.5 | 4 | 4 | 4.5 |
| tazobactam + | | | | | | | | | | |
| Cefepime | | | | | | | | | | |
| ceftazidime + | 3 | 4 | 3.5 | 3.5 | 4 | 4 | 5.5 | 5.5 | 5.0 | 5.5 |
| Tobramycin | | | | | | | | | | |
| piperacillin- | 3.5 | 4 | 3.5 | 3 | 4 | 4.5 | 5 | 5 | 5 | 4.5 |
| tazobactam + | | | | | | | | | | |
| Tobramycin | | | | | | | | | | |
| Mean | 3.2 | 3.6 | 3.5 | 3.6 | 3.7 | 4.2 | 4.7 | 4.5 | 4.4 | 4.6 |

MM—mixed medium (Columbia agar + Pepted Meat agar);
LB—Luria Broth agar;
PMA—Pepted Meat agar;
CA—Columbia agar;
SA—Schaedler agar

Conclusion. Analyzing the presence of bacterial growth (with Method (Algorithm) #1), by comparing the appearance (progression) of microbial growth through the time within the single well supplemented with antibiotic, enables quicker selection of effective antibiotics compared with the method (algorithm) #2), when the presence of bacterial growth is evaluated by analyzing of the presence of bacterial growth in wells supplemented with antibiotics, when compared with control antibiotic-free wells.

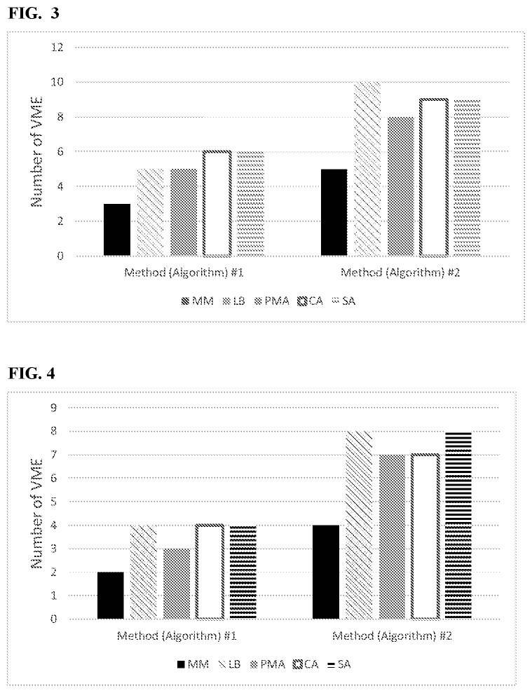

Next, we analyzed, how accurate are the results obtained by monitoring early signs of microbial growth in terms for their progression to growth during the first 24 h. We were particularly interested in possible very major errors (VME), defined as when according to the test system, antibiotic was effective (no microbial growth) at the first timepoints of 2.5-5 h, but then, microbial growth progressed with the appearance of microcolonies during the subsequent cultivation for 24 h. Data are presented in FIGS. 3, 4.

Conclusion: As it is seen, the use of using Method (Algorithm) #1 comparing the signs of bacterial growth in each well supplemented with antibiotic(s) at certain timepoint, with the signs of bacterial growth in the same well at previous time point, provides more accurate data analysis with a significantly less amount of very major errors. The use of Mixed Media provides more accurate results compared to other media. The use of microscope increases the accuracy of data received.

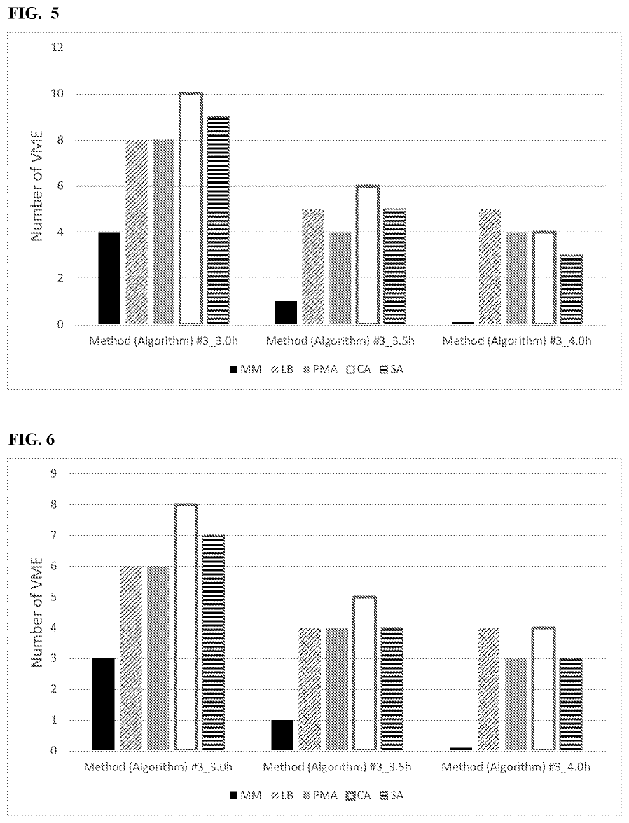

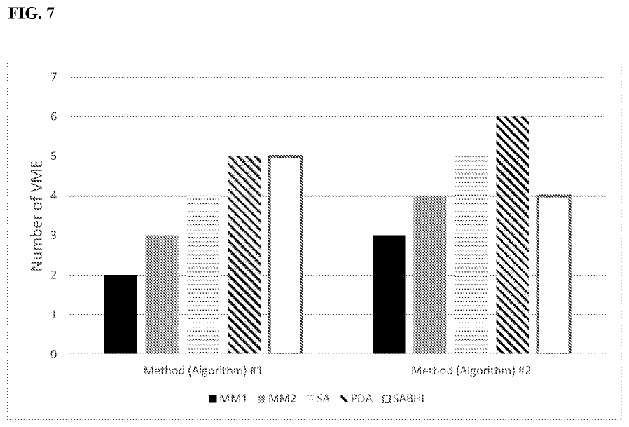

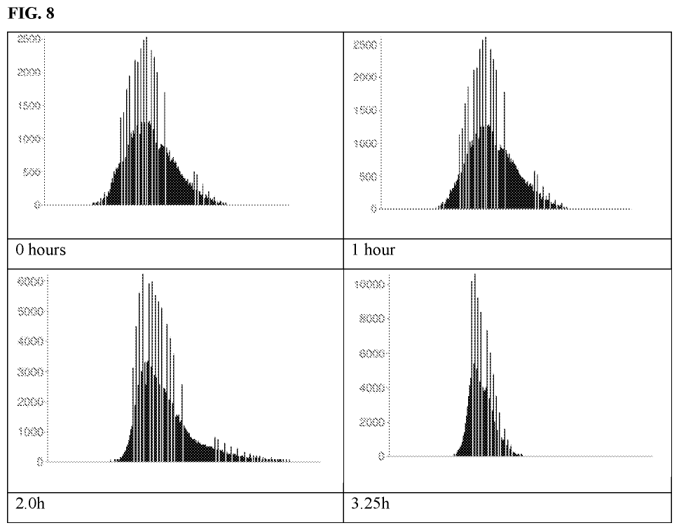

We next monitored, how does the analysis of the absence of bacterial growth at different fixed timepoints is accurate in terms for the following progression of microbial growth during subsequent cultivation for 24 h (FIG. 5).

Conclusion: As it is seen, the use of Method (Algorithm) #3, when we monitor the absence of bacterial growth provides a reliable results at the timepoints faster than that of

Method (Algorithm) #2. The use of Mixed Media provides more accurate results compared to other media. The use of microscope insignificantly increases the accuracy of data received.

### Example 4. Novel Media Composition Used for Diverse, Quick Cultivation of Fungi

The novel medium (Mixed Medium 1) for fugal cultivation was prepared as follows: the following weighed portions of the medium components were prepared (for 1 liter): potato decoction 200 g, corn flour tincture 50 ml, oat flour decoction 350 ml, potato-carrot decoction 300 ml, Sucrose, 30 g, Cellobiose, 20 g, Yeast extract, 4 g, Maltose, 40 g, NaNO3—2,0 g, K2HPO4—1,0 g, MgSO4—0,5 g, KCl—0,5 g, FeSO4—1 g, Zn SO4—0,1g, MnCl2-0,1 g, Twin80—10 ml.

The Mixed Medium 1 (MM1) was sterilized by autoclaving at 1.0 atm at 120. C for 20 minutes. To assess diversity and speed of fungal growth, different fungi from the collection of Human Microbiology Institute were placed on the MM1 or Sabouraud agar and sights of the visual fungal growth in less than 6 hours was assessed.

The results of the comparison of claimed nutrient medium MMI with Sabouraud agar are given in table 5.

TABLE 5
Visible fungal grown on nutrient media

Visible fungal grown on
nutrient media in <6 h

| Fugi (spp) | Mixed Media 1 | Sabouraud agar |
|---|---|---|
| Candida | + | − |
| Aspergillus | + | + |
| Mucor | + | − |
| Trichophyton | + | − |
| Blastomyces | + | − |
| Cryptococcus | + | − |
| Pneumocystis | + | − |
| Paracoccidioides | + | − |
| Histoplasma | + | − |
| Coccidioides | + | − |
| Talaromyces | + | − |
| Sporothrix | + | − |
| Fusarium | + | + |
| Malassezia | + | − |
| Microsporum | + | − |
| Saccharomyces | + | − |
| Saprolegnia | + | − |
| Helmintosporium | + | − |
| Penicillium | + | − |
| Eurotium | + | − |
| Chrysosporium | + | − |
| Cladosporium | + | − |
| Alternaria | + | − |

Analysis of the results showed that the declared nutrient medium MMI allows the cultivation of a maximum number of fungi within less than 6 hours compared to other broadly used nutrient media used for fungal cultivation.

### Example 5. Effect of Data Analysis Algorithm on Speed and Accuracy of Antifungal Antibiotic Selection

Oral swabs (n=6) from patients with confirmed oral candidiasis caused by C. albicans, non-albicans Candida spp. Aspergillus spp., were dissolved with PBS and 0.1 uL were plated on 12 well plate test system filled with Mixed Medium 1 (see above), Mixed Medium 2 (Sabouraud agar+Potato Dextrose Agar+vitamins B12+B7) (MM2), Sabouraud agar (SA), Potato Dextrose Agar (PDA), Sabouraud's Heart Infusion (SABHI) agar. 11 wells were filled with media supplemented with antibiotics (taken at mean concentration of each antibiotic achieved in lungs at different timpoints post drug administration)—Nystatin, Amphotericin B, Clotrimazole, Fluconazole, Isavuconazole, Terbinafin, Posaconazole, Voriconazole, Anidulafungin, Caspofungin, Micafungin. One well was antibiotic free.

Plates was incubated at 30 C and the analysis of the presence/absence of microbial growth was monitored every 15 minutes from 9 h to 5 h, then hourly from 5 h to 8 h and then at 18 and 24 h post plating with a naked eye.

Three methods (algorithms) how to analyze fungal growth were used.

Method (Algorithm) #1. Lab technician, analyzed the appearance of fungal growth by comparing the signs of fungal growth in each well supplemented with antibiotic(s) at certain timepoint, with the signs of funal growth in the same well at previous time point(s).

Method (Algorithm)#2. Lab technician analyzed the appearance of fungal growth by comparing the signs of fungal growth in each well supplemented with antibiotic(s) with fungal growth in antibiotic-free control

Method (Algorithm) #3. Lab technician analyzed the absence of fungal growth in the wells in fixed time period of 3.5 h; 5 h and 24 h. The results are shown in Table 6.

TABLE 6
Analysis of the signs of fungal growth following naked eye examination
in the samples that were resistant to a certain antibiotic

| | Mean hour at which fungal growth became visible | | | | | | | | | |
|---|---|---|---|---|---|---|---|---|---|---|
| | on the wells supplemented with a certain antibiotic | | | | | | | | | |
| | Method (Algorithm) #1 | | | | | Method (Algorithm) #2 | | | | |
| Antibiotic | MM1 | MM2 | SA | PDA | SABHI | MM1 | MM2 | SA | PDA | SABHI |
| No Antibiotic | 4 | 5 | 6 | 6 | 6.5 | 5 | 5.5 | 5.5 | 8 | 7.5 |
| Nystatin | 4 | 4.5 | 6 | 5 | 5.5 | 4.5 | 6 | 6 | 6.5 | 7.5 |
| Amphotericin B | 3 | 5 | 6.5 | 4.5 | 6 | 5 | 5 | 7 | 7.5 | 8 |
| Clotrimazole | 3.5 | 4.5 | 5 | 7.5 | 6.5 | 4.5 | 6.5 | 7.5 | 8.5 | 6.5 |
| Fluconazole | 4 | 4.5 | 6.5 | 5 | 7.5 | 5.5 | 5.5 | 7 | 6.5 | 8 |
| Isavuconazole | 5 | 4.5 | 6.5 | 6 | 7 | 6 | 5.5 | 7.5 | 7.5 | 8.5 |
| Terbinafin | 4.5 | 5 | 5 | 5 | 5 | 4.5 | 5.5 | 6.5 | 8 | 6.5 |
| Posaconazole | 3.5 | 4.5 | 5 | 5 | 5.5 | 4.5 | 5 | 7 | 8.5 | 7 |
| Voriconazole | 4 | 4.5 | 4.5 | 5.5 | 5.5 | 4.5 | 4.5 | 8 | 6.5 | 7 |
| Anidulafungin | 4 | 5 | 5.5 | 6.5 | 6.5 | 5.5 | 4.5 | 6.5 | 7.5 | 8.5 |
| Caspofungin | 3.5 | 5 | 5.5 | 6 | 7 | 6 | 5.5 | 7.5 | 8 | 8.5 |
| Micafungin | 4.5 | 5 | 6 | 6 | 5 | 4.5 | 5.5 | 8 | 7.5 | 6.5 |
| Mean | 3.96 | 4.75 | 5.59 | 5.67 | 6.13 | 5.00 | 5.38 | 7.00 | 7.54 | 7.50 |

Conclusion. Analyzing the presence of fungal growth (with Method (Algorithm) #1), by comparing the appearance (progression) of fungal growth through the time within the single well supplemented with antibiotic, enables quicker selection of effective antibiotics compared with the method (algorithm) #2), when the presence of fungal growth is evaluated by analyzing of the presence of bacterial growth in wells supplemented with antibiotics, when compared with control antibiotic-free wells at the same time period.

Next, we analyzed, how accurate are the results obtained by monitoring early signs of fungal growth in terms for their progression to growth during the first 24 h. We were particularly interested in possible very major errors (VME), defined as when according to the test system, antibiotic was effective (no bacterial growth) at the first timepoint of 4-8 h, but then, it progressed to the appearance of growth during the subsequent cultivation for 24 h. Data are presented in FIG. 7.

Conclusion: As it is seen, the use of using Method (Algorithm) #1, provides more accurate data analysis with a significantly less amount of very major errors. The use of mixed media 1 and mixed media 2, provide more accurate results compared to other media.

### Example 6. The Use of Computer-Generated Imagery to Analyze Presence of Microbial Growth

The same sputum samples as used before, from patients with VAP were resuspended in PBS and 0.2 uL were plated with Eppendorf single channel pipette on 12 well plate test system filled with mixed medium (Columbia agar+Pepted Meat agar+10% erythrocytes) without of adding antibiotics.

Plates was incubated at 37 C and the analysis of the presence/absence of microbial growth was monitored every 15 minutes from 0 h to 5 h with a (i) naked eye or (ii) images were taken on iPhoneX at the regular settings. For both (i) and (ii) we used fixed settings with the fixed distance of 10 cm from an eye/camera, and an angle of 60°. Images taken at the digital camera were converted to a greyscale and then, image histogram was generated with the online software https://www.dcode.fr/image-histogram . The results are shown in Table 7 and FIG. 8.

TABLE 7
Analysis of the signs of bacterial growth following naked
eye examination or analysis with a digital algorithm

| | | | | Mean hour at which | | |
|---|---|---|---|---|---|---|
| | Mean hour at which | | | bacterial growth became | | |
| | bacterial growth became | | | visible according to greyscale | | |
| | visible with a naked eye | | | image histogram analysis | | |
| | Sample | Sample | Sample | Sample | Sample | Sample |
| Well | 1 | 2 | 3 | 1 | 2 | 3 |
| 1 | 3 | 3.25 | 3.25 | 2.5 | 2.5 | 2.5 |
| 2 | 3 | 3.25 | 3.25 | 2.75 | 2.5 | 2 |
| 3 | 3 | 3 | 3.5 | 2.75 | 2.75 | 3 |
| 4 | 3 | 3 | 3.25 | 3 | 2.5 | 2.75 |
| 5 | 3 | 3.25 | 3.25 | 2.5 | 2.5 | 2 |
| 6 | 3 | 3 | 3.25 | 2 | 2.25 | 2.5 |
| 7 | 3.25 | 3.5 | 3.5 | 2 | 2.25 | 2.5 |
| 8 | 3 | 3 | 3.25 | 2.75 | 2.25 | 2.75 |
| 9 | 3 | 3.25 | 3.25 | 2.75 | 3 | 3 |
| 10 | 3 | 3 | 3.5 | 2.75 | 2.5 | 3 |
| 11 | 3.25 | 3 | 3.5 | 2.5 | 2.75 | 2.5 |
| 12 | 3 | 3 | 3.25 | 2.75 | 2.5 | 2.75 |
| Total | 3.04 | 3.125 | 3.33 | 2.58 | 2.52 | 2.60 |

As it is seen the use of image analysis software provides a faster and more reliable results compared with naked eye.

### Example 7. Use of Foil or Mu-metal Shielding for the Regulation of Bacterial Types that give Growth on Selected Nutrient Media

We next studied, could we alterations of magnetic field affect the diversity of bacteria grown on nutrient media. For that we plated 50 uL of biosample (sputum) to the medium composed of Columbia and Pepted Meat agars and incubated at different timepoints at 37 C under normal or altered magnetic/electriomagnetic field. We modulated altered magnetic field by growing bacteria in Mu-shield material or by plating Petri dish placed in foil.

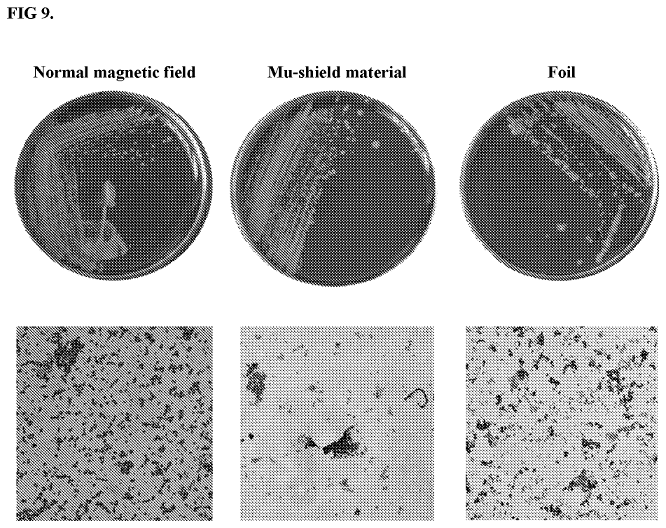

Microscopic experiments were performed using a Nikon Eclipse Ti (Nikon Plan Fluor ×100/1.30 Oil Ph3 DLL and Plan Apo×100/1.40 Oil Ph3 objectives) microscope. Bacterial morphology was determined by staining cell membranes with methylene blue or Gram staining (Sigma). The results are shown in table 8 and FIG. 9.

TABLE 8
Microscopy study of bacterial diversity following cultivation
in different magnetic and/or electromagnetic fields

| | Proportion of bacteria | | |
|---|---|---|---|
| | Normal magnetic | Mu-shield | |
| Bacteria | field | material | Foil |
| Gram positive cocci | +++++ | +++ | +++ |
| Gram negative rods | 0 | ++ | ++ |

It was clearly seen that the diversity of bacteria was significantly different following alterations of magnetic and/or electromagnetic fields and that alterations of magnetic and/or electromagnetic field with non-limiting examples for the use of foil and/mu-materials can be used for the modulation of microbial diversity.

### Example 8. Effect of the Nucleases on the Speed of Bacterial Growth

Endotracheal aspirate (n=3), sputum (n=3), BAL (n=2), wound swab (n=3) from patient with pneumonia was dissolved with PBS and 0.1 uL were plated on 12 well plate test system filled with mixed medium (Columbia agar+Pepted Meat agar+10% erythrocytes). 9 wells were filled with media supplemented with nucleases (DNase I, RNase, DNase+RNase) taken at different concentrations up to 100 ug/mL.

Plates was incubated at 37 C and the analysis of the presence/absence of microbial growth was monitored every 15 minutes from 0 h to 5 h, then hourly from 5 h to 8 h and then at 18 and 24 h post plating with a naked eye or with an AmScope microscope on 20× magnification with 12 mp camera. The results are shown in Table 9.

TABLE 9
Analysis of the signs of bacterial growth following in
the samples that were resistant to a certain antibiotic
Mean hour at which bacterial growth became visible
on the wells supplemented with a certain antibiotic

| | |
|---|---|
| Naked eye examination | Microscopic examination |

| CC | D | R | D + R | CC | D | R | D + R |
|---|---|---|---|---|---|---|---|
| 3 | 2.5 | 2.75 | 2.75 | 3 | 2.5 | 2.5 | 2.5 |

CC—control;
D—DNase;
R—RNase;
D + R—DNase + RNase

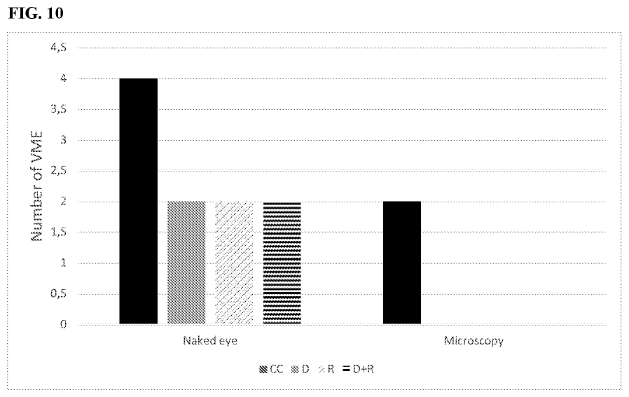

Next, we analyzed, how accurate are the results obtained by monitoring early signs of microbial growth in terms for their progression to growth during the first 24 h. We were particularly interested in possible very major errors (VME), defined as when according to the test system, antibiotic was effective (no bacterial growth) at the first timepoint of 2.5-5 h, but then, it progressed to the appearance of growth during the subsequent cultivation for 24 h (FIG. 10).

Conclusion. Cultivation of biosamples on the medium supplemented with nucleases and/or treatment of the biosample with different concentrations of nucleases enables faster growth of bacteria and more precise analysis of the growth at early stage of growth, with a lower number of errors.

### Example 9. Effect of the Alteration of Magnetic and/or Electromagnetic Fields on the Bacterial Diversity and Microbial Sensitivity in the Presence of Antimicrobial Agents

We next studied, could the alterations of magnetic field affect the diversity of bacteria grown on nutrient media. For that we plated 50 uL of biosample (sputum) to the medium composed of Columbia and Pepted Meat agars and incubated at different timepoints at 37 C under normal or altered magnetic/electromagnetic field. We modulated altered magnetic field by growing bacteria in Mu-shield material or by plating Petri dish placed in foil. Antibiotics were added to the medium directly as the mean concentrations achievable at the site of infection during 4 h, post administration

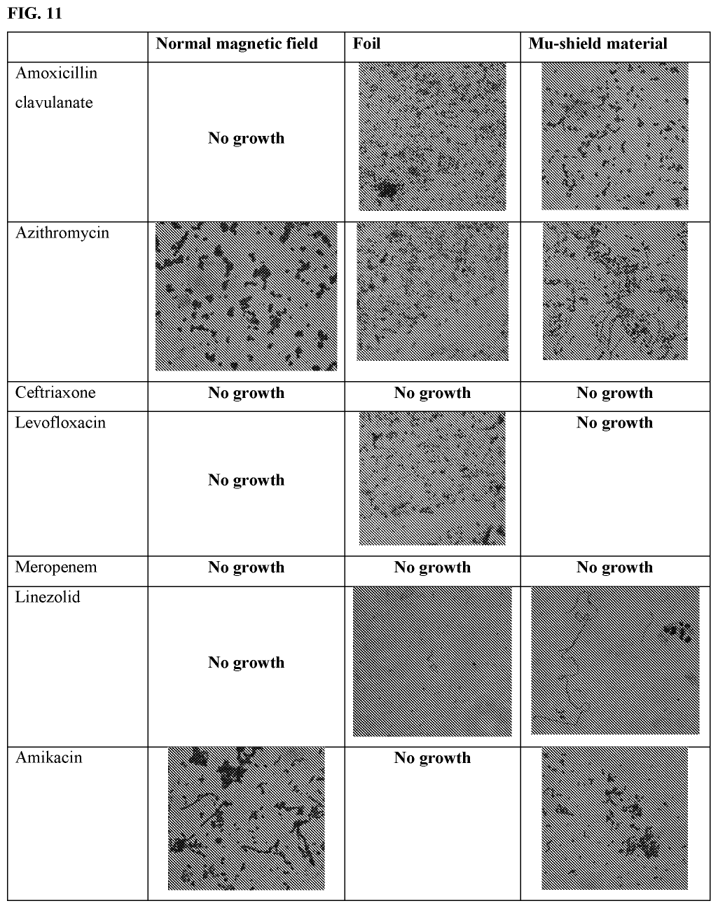

Antibiotic efficacy was determined by the presence or absence of bacterial growth detected with naked eye within 24 h of cultivation. Microscopic experiments were performed using a Nikon Eclipse Ti (Nikon Plan Fluor×100/1.30 Oil Ph3 DLL and Plan Apo×100/1.40 Oil Ph3 objectives) microscope. Bacterial morphology was determined by staining cell membranes with methylene blue or Gram staining (Sigma). The results are shown in tables 10, 11 and FIG. 11.

TABLE 10
Effect of different magnetic and/or electromagnetic
fields on the efficacy of antibiotics

| | Normal magnetic | | Mu-shield |
|---|---|---|---|
| | field | Foil | material |
| Amoxicillin clavulanate | S | R | R |
| Azithromycin | R | R | R |
| Ceftriaxone | S | S | S |
| Levofloxacin | S | S | S |
| Meropenem | S | S | S |
| Linezolid | S | R | R |
| Amikacin | R | S | R |
| Vancomycin | R | R | S |
| Clindamycin | R | R | R |
| Cefepime | S | S | S |

R—resistant (i.e. absence of bacterial growth);
S—Sensitive (i.e. presence of bacterial growth)

TABLE 11
Microscopy study of bacterial diversity following cultivation
in different magnetic and/or electromagnetic fields

| | Proportion of bacteria | | |
|---|---|---|---|
| Bacteria | Normal magnetic field | Foil | Mu-shield material |
| Gr+ micrococci | + | + | + |
| Gr+ diplococci | − | + | − |
| Gr+ streptococci | + | − | + |
| Gr+ rods | + | + | + |
| Gr+ coryneform | − | + | − |
| Gr− rods | − | + | + |
| Gr− cocci | − | − | + |
| Gr− filiform | − | + | + |

Conclusion. It is clearly seen that growth of bacteria in different magnetic and/or electromagnetic fields modulated with Mu-metal or foil differentially affected the diversity of microorganisms that give growth on the medium. It also enabled the selection of different types of bacteria and overgrowth certain bacterial species. The magnetic/electromagnetic shielding with Mu-metal increases the diversity of Gram-negative rods in the mix, and the use of foil increase diversity of Gram-negative and Gram-positive bacteria in the mix and the number of phenotypes that give growth.

### Example 10. Effect of the Alteration of Magnetic and/or Electromagnetic Fields on the Eucaryotic Cell Growth and Characteristics

We next studied, could the alterations of magnetic field affect the eucaryotic cells growth. For that a total of 5×105 CHO-K1 cells from 48 h monolayer, were grown on Petri dishes with DMEM culture medium (Gibco) supplemented with 10% fetal bovine serum and 1% penicillin/streptomycin (Invitrogen) at 37 C in a humidified 5% CO2 for 24 h (Sanyo MCO-19AIC).

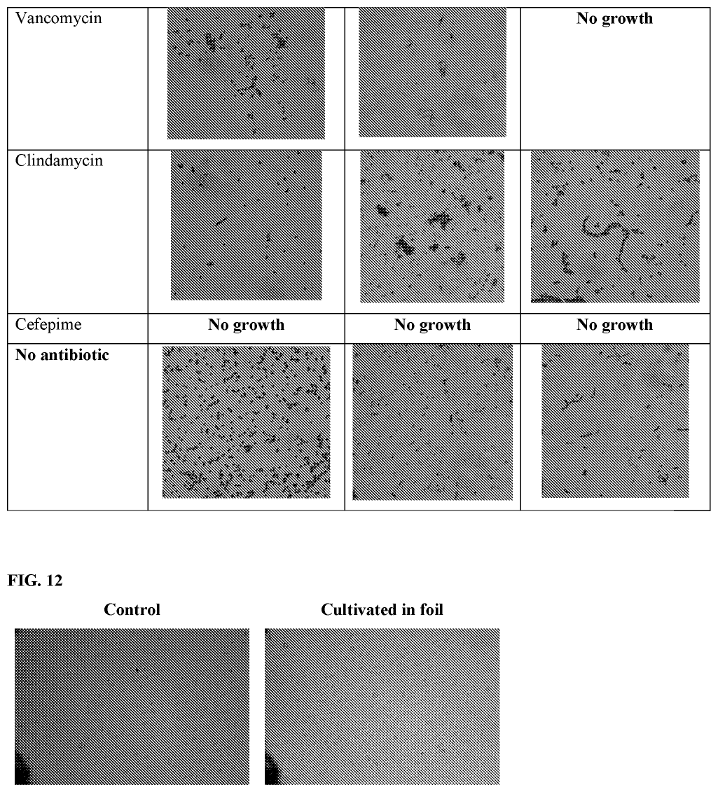

We modulated altered magnetic field by growing bacteria in foil boxes made of 50 microns foil. Microscopy was done with Axiovert 40 CFL (Zeiss). The results are shown in Table 12 and FIG. 12.

TABLE 12
Use of foil to increase cell number

| | Control | Cultivated in foil |
|---|---|---|
| Cell number in three | 256 | 395 |
| microscope fields | | |

This data clearly shows that the use of foil increases cells' synthetic activity and cell number

### Example 11. Use of Mu-Metal to Modulate Eucaryotic Cells Growth

To access the role of Mu-metal in acceleration of eucaryotic cells growth we used C. glabrata MR-V32. Standard inoculum for yeast testing was 2.5×10e3 CFU/ml, fungi were cultivated for 24 h of incubation at 35° C. in Sabouraud broth. Control fungi were grown in Sanyo incubator (MCO-19AIC), or placed in in-house made Mu-metal envelope placed in Sanyo incubator (MCO-19AIC), or placed in in Mu-metal box placed in Sanyo incubator (MCO-19AIC). OD600 at baseline, after 6 and 24 h of growth was accessed using NanoDrop OneC spectrophotometer (ThermoFisher Scientific, Waltham, MA, USA). The results are shown in Table 13.

TABLE 13
Mu-metal to modulate eucaryotic cells growth

| | OD 600 | | | | | | | | |
|---|---|---|---|---|---|---|---|---|---|
| | Control | | | Mu-envelope | | | Mu-box | | |
| | 0 h | 6 h | 24 h | 0 h | 6 h | 24 h | 0 h | 6 h | 24 h |
| C. glabrata | 0.025 | 0.992 | 4.345 | 0.03 | 1.345 | >10000 | 0.025 | 2.358 | >10000 |
| MR-V32 | | | | | | | | | |

These data shows that the cultivation of eucaryotic cells in mu-metal increases cells' synthetic activity and cell number

### Example 12. The Use of a Novel Incubators for Growth of Microorganisms with a Unique Properties and/or Modulation of the Real-Life Conditions for Microbial Growth as at the Site of Infections

We compared the properties of microorganisms directly at the site of infection, when they were cultivated is a specially developed incubator, and in commercially available incubators.

As a commercially available incubators we used Sanyo iron and nickel chamber—ferromagnetic properties, increases geomagnetic field, and Heracell 150i CO2 incubator (Thermo Fisher Scientific) with Copper Chamber—diamagnetic properties, decreases geomagnetic field

We developed four types of specially developed incubators (Bio adjustable Tetz incubators):

Prototype 1—incubator with added Mu-metal and foil layers separated from each other with diamagnetic layers

Prototype 2—incubator with added Mu-metal and foil layers separated from each other with diamagnetic layers

Prototype 3—incubator with added Mu-metal to the chamber

Prototype 4—incubator with added foil layers separated from each other with diamagnetic plastie layers incubator with added Mu-metal to the chamber

Biosample (saliva) was plated to the medium composed of Columbia and Pepted Meat agar, in a 90 mm Petri dish (Corning).

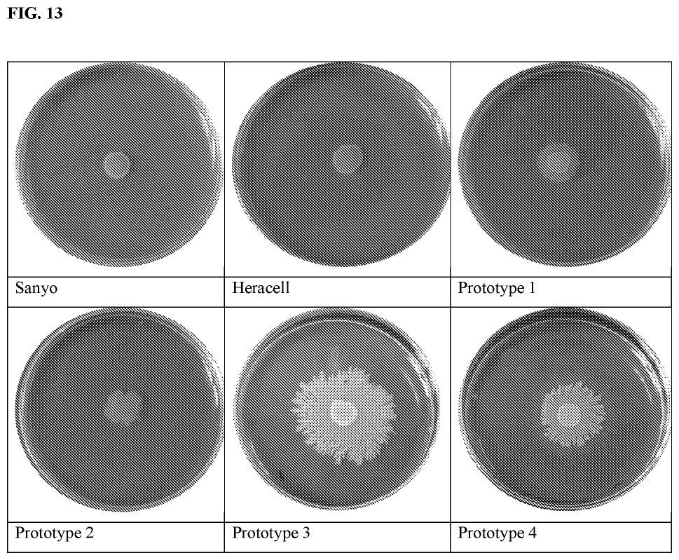

The results are shown in FIG. 13.

We unexpectedly found that the alteration of geomagnetic field with the incubator differentially affected microbial growth. The use of different incubators (Bio adjustable Tetz incubators) differently affect geomagnetic field of the Earth, external magnetic fields, personal electromagnetic field that is generated by microorganisms during their growth, allows to obtain microorganisms with different properties, i.e. different synthetic activity.

### Example 13. The Use Mu-Metal-Containing Devices to Monitor Environmental Conditions, Radiation and Ecology

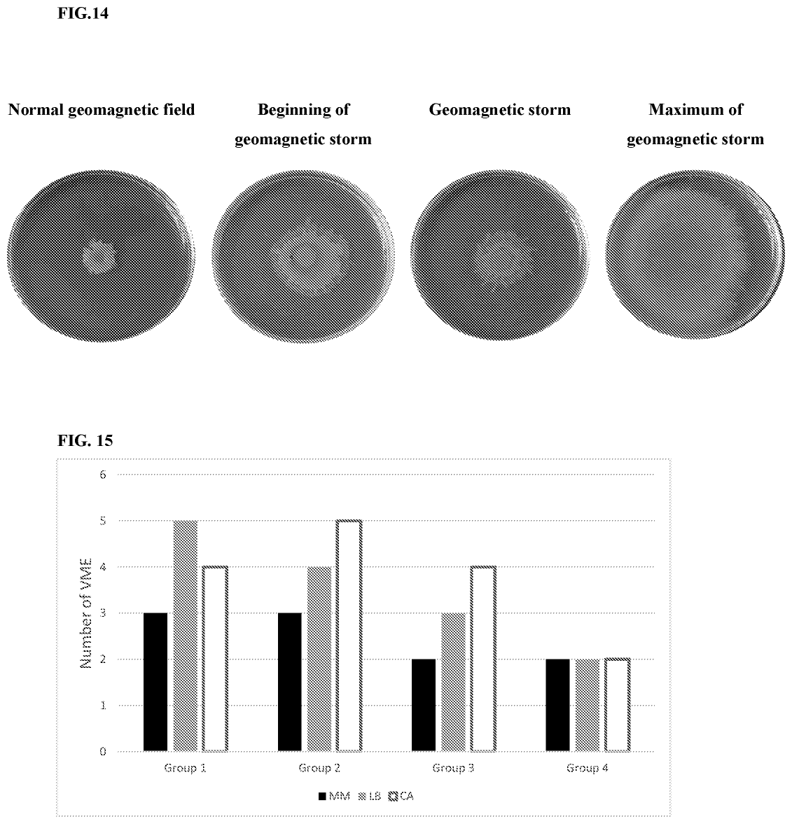

We used Mu-metal made box as a device to monitor environmental, weather and geomagnetic conditions. For that, daily we plated microorganisms (including sporeforming) on the surface of Columbia agar on 90 mm glass Petri dishes, that were placed in Mu-metal boxes and cultivated for 24 h at 37C. We analyzed alterations of biofilm morphology and aligned these alterations with the geomagnetic storms. The results are shown in FIG. 14.

As it seen, by cultivating microorganisms in Mu-metal it is possible to detect geomagnetic storms and other alterations and disturbance of the magnetosphere.

### Example 14. Supplements Added to Culture Media to Control the Growth of Specific Groups of Microorganisms

We next studied, could we regulate bacterial diversity grown from the bio samples with different compounds including transcriptase inhibitors, protease inhibitors, recombinase/integrase inhibitors, nucleases. For that we plated 10 uL of biosamples (sputum, BAL, wound swab) to the medium composed of Columbia and Pepted Meat agars supplemented with 10% erythrocytes and incubated at different timepoints at 37 C (table 14).

TABLE 14
Compounds added to the media and/or used to treat the biosample prior to plating

| Group | Compound | Concentration | |
|---|---|---|---|
| 1 | 2-cyclopropyl-7-methyl-2,4,9,15- | 0.5 | ug/mL |
| | tetrazatricyclo[9.4.0.0^{3,8}]pentadeca-1(11),3,5,7,12,14-hexaen- | | |
| | 10-one; hydrate | | |
| 2 | 2-cyclopropyl-7-methyl-2,4,9,15- | 100 | ug/mL |
| | tetrazatricyclo[9.4.0.0^{3,8}]pentadeca-1(11),3,5,7,12,14-hexaen- | | |
| | 10-one; hydrate | | |
| 3 | 4-[6-amino-5-bromo-2-(4-cyanoanilino)pyrimidin-4-yl]oxy- | 0.5 | ug/mL |
| | 3,5-dimethylbenzonitrile | | |
| 4 | 4-[6-amino-5-bromo-2-(4-cyanoanilino)pyrimidin-4-yl]oxy- | 100 | ug/mL |
| | 3,5-dimethylbenzonitrile | | |
| 5 | 4-amino-1-[(2R,5S)-2-(hydroxymethyl)-1,3-oxathiolan-5- | 0.5 | ug/mL |
| | yl]pyrimidin-2-one | | |
| 6 | 4-amino-1-[(2R,5S)-2-(hydroxymethyl)-1,3-oxathiolan-5- | 100 | ug/mL |
| | yl]pyrimidin-2-one | | |
| 7 | [(2R)-1-(6-aminopurin-9-yl)propan-2-yl]oxymethylphosphonic | 0.5 | ug/mL |
| | acid | | |
| 8 | [(2R)-1-(6-aminopurin-9-yl)propan-2-yl]oxymethylphosphonic | 100 | ug/mL |
| | acid | | |
| 9 | 1-[(2R,3R,4S,5R)-3,4-dihydroxy-5-(hydroxymethyl)oxolan-2- | 0.5 | ug/mL |
| | yl]-1,2,4-triazole-3-carboxamide | | |
| 10 | 1-[(2R,3R,4S,5R)-3,4-dihydroxy-5-(hydroxymethyl)oxolan-2- | 100 | ug/mL |
| | yl]-1,2,4-triazole-3-carboxamide | | |
| 11 | sodium; 2-amino-9-(2-hydroxyethoxymethyl)purin-6-olate | 0.5 | ug/mL |
| 12 | sodium; 2-amino-9-(2-hydroxyethoxymethyl)purin-6-olate | 100 | ug/mL |
| 13 | Potassium Orotate | 0.5 | ug/mL |
| 14 | Potassium Orotate | 100 | ug/mL |
| 15 | [(1S,4R)-4-[2-amino-6-(cyclopropylamino)purin-9- | 0.5 | ug/mL |
| | yl]cyclopent-2-en-1-yl]methanol | | |
| 16 | [(1S,4R)-4-[2-amino-6-(cyclopropylamino)purin-9- | 100 | ug/mL |
| | yl]cyclopent-2-en-1-yl]methanol | | |
| 17 | N-[2-[4-[(4-fluorophenyl)methylcarbamoyl]-5-hydroxy-1- | 0.5 | ug/mL |
| | methyl-6-oxopyrimidin-2-yl]propan-2-yl]-5-methyl-1,3,4- | | |
| | oxadiazole-2-carboxamide | | |
| 18 | N-[2-[4-[(4-fluorophenyl)methylcarbamoyl]-5-hydroxy-1- | 100 | ug/mL |
| | methyl-6-oxopyrimidin-2-yl]propan-2-yl]-5-methyl-1,3,4- | | |
| | oxadiazole-2-carboxamide | | |
| 19 | DNase | 0.5 | ug/mL |
| 20 | DNase | 100 | ug/mL |
| 21 | RNase | 0.5 | ug/mL |
| 22 | RNase | 100 | ug/mL |
| 23 | DNase + RNase | 0.5 | ug/mL |
| 24 | DNase + RNase | 100 | ug/mL |
| 25 | Proteinase | 0.1 | ug/mL |
| 26 | Proteinase | 10 | ug/mL |

The efficacy of the compound to modulate bacterial diversity was determined by the presence or absence of bacteria with certain morphological properties after 18 h of cultivation.

Microscopic experiments were performed using a Nikon Eclipse Ti (Nikon Plan Fluor ×100/1.30 Oil Ph3 DLL and Plan Apo×100/1.40 Oil Ph3 objectives) microscope. Bacterial morphology was determined by staining cell membranes with methylene blue or Gram staining. The results are shown in Table 15.

TABLE 15
Effects of supplements added to culture media to control
the growth of specific groups of microorganisms

| | Bacteria* | | |
|---|---|---|---|
| | Gram positive non- | Gram positive | Gram |
| | sporeforming | sporeforming | negative |
| Group | bacteria | bacteria | rods |
| Control | +++ | +++ | +++ |
| 1 | +++++ | +++ | +++ |
| 2 | +++++ | +++ | +++ |
| 3 | +++++ | +++ | +++ |
| 4 | +++++ | +++ | +++ |
| 5 | +++++ | +++ | +++ |
| 6 | +++++ | +++ | +++ |
| 7 | +++++ | +++ | +++ |
| 8 | +++++ | +++ | +++ |
| 9 | +++++ | +++ | +++ |
| 10 | +++++ | +++ | +++ |
| 11 | +++++ | +++ | +++ |
| 12 | +++++ | +++ | +++ |
| 13 | +++++ | +++ | +++ |
| 14 | +++++ | +++ | +++ |
| 15 | +++ | +++ | +++++ |
| 16 | +++ | +++ | +++++ |
| 17 | +++ | +++ | +++++ |
| 18 | +++ | +++ | +++++ |
| 19 | +++++ | +++ | +++ |
| 20 | +++++ | +++ | +++ |
| 21 | +++++ | +++ | +++ |
| 22 | +++++ | +++ | +++ |
| 23 | +++ | +++ | +++++ |
| 24 | +++ | +++ | +++++ |
| 25 | +++ | +++ | +++++ |
| 26 | +++ | +++ | +++++ |

The diversity of certain bacteria were presented in “+” where “+++” marks the diversity of certain bacterial type in control and “+++++” indicates an increased diversity of bacterial type.

It was clearly seen that the diversity of bacteria was significantly different following the use of different compounds listed in this example as well as nucleases and proteinases. The use of compounds listed enabled the selection of different types of bacteria and overgrowth of certain bacterial species.

### Example 15. Use of Test System to Select Previously Unculturable Bacteria

The swab from the tongue of the patient with Alzheimer's disease was plated on dense media: (i) Columbia and Pepted Meat agars or (ii) LB agar or (iii) Columbia agar supplemented or not supplemented with 5% sheep erythrocytes, antibiotics, nucleases.

Nucleases added to agars were: DNase I, RNase (all Sigma-Aldrich) or their mix taken at 100 ug/mL. Probes were cultivated at 37 C for 24 h. Colonies of different morphotypes were replated to the appropriate agar medium with or without antibiotics or nucleases for the next 24 h at 37. This cycle was repeated several times to obtain monospaces colonies according to microscopy examination. The results are shown in Table 16.

TABLE 16
Number of different bacterial species isolated

| | | | ER + | ER − | ER + | ER − | ER + | ER − | ER + | ER − |
|---|---|---|---|---|---|---|---|---|---|---|
| Media | ER+ | ER− | LEV+ | LEV+ | D+ | D+ | R+ | R+ | DR+ | DR+ |
| COL + PEP | 8 | 6 | 4 | 2 | 8 | 8 | 9 | 9 | 9 | 8 |
| LB | 5 | 4 | 2 | 0 | 6 | 5 | 6 | 6 | 5 | 5 |
| COL | 6 | 4 | 0 | 0 | 7 | 7 | 7 | 6 | 7 | 5 |

COL + PEP Columbia and Pepted Meat agar,
LB—LB agar,
COL—Columbia agar,
agent added (+),
agent not added (−),
Erythrocytes (ER),
Levofloxacin (LEV),
DNase I (D),
RNase (R),
DNase I + RNase (DR)

As it seen, different excipients added to the medium differently affected the growth of bacteria and some of them enabled the growth of more diverse range of microorganisms.

We next identified bacterial species, that were unique (based on morphology of the colonies or microscopy) compared between probes having or not having some excipients. The number of bacteria identified as “unique” are listed in table 17.

TABLE 17
Unique bacterial species identified with proposed method

| | ER + | ER − | ER + | ER − | ER + | ER − | ER + | ER − |
|---|---|---|---|---|---|---|---|---|
| Media | LEV+ | LEV+ | D+ | D+ | R+ | R+ | DR+ | DR+ |
| COL + | 2 | 1 | 1 | 0 | 1 | 1 | 1 | 0 |
| PEP | | | | | | | | |
| LB | 1 | 0 | 1 | 0 | 1 | 1 | 1 | 0 |
| COL | 0 | 0 | 1 | 1 | 1 | 1 | 1 | 0 |

As it is seen the adding of different compounds enabled the growth of bacteria that were present in the biosample, but were not growing according to a standard method. After that genomic DNA was extracted using a genomic DNA purification kit (QIAamp). The 16S rRNA gene was amplified with the universal bacterial primers 27F and 1492R and assembled using SeqMan v7 softwar. The 16S rRNA gene of Streptococcus oraculs sp.nov. possesses 95% sequence identity with S. pneumoniae and 94% sequence identity with S. mitis.

Paired-end libraries (300-bp length) were prepared using a TruSeq DNA sample prep kit and then sequenced using a HiSeq 2500 instrument (Illumina, USA). Samples underwent library preparation and sequencing according to the manufacturer's instructions.

Raw sequence reads were cleaned with Trimmomatic (v0.38) to remove Illumina adapters and trimmed within a sliding 4-base window, cutting when the average quality per base dropped below 15. Contaminating human sequences were removed by alignment to the human reference genome (GRCH38) with Bowtie 2 (v2.3.4.2), collecting only unaligned read pairs. A draft genome was assembled using SPAdes v3.7.1 with default parameters. The assembled 20 contigs had an average kmer coverage of 150-fold, with a total length of 1,970,714 bp, a GC content of 41.6%. An in silico DNA-DNA hybridization was carried out using the Genome-to-Genome Distance Calculator.

The results revealed that the S. oraculs sp.nov. genome was distinct from the genomes of representative strains of related species (i.e., S. pneumoniae, and S. mitis, with similarity values of 24.60 and 32.20%, respectively) and were below the 70% cutoff for DNA-DNA hybridization.

### Example 17. Use of the Method Described to Select Effective Antibiotics with as Narrow as Possible Spectrum of Activity Against PP

The analysis of antibiotics selected as effective was analyzed by the disappearance of Primary Pathogen Pseudomonas aeruginosa following adding of antibiotics suggested as effective with proposed method vs direct plating of biosample vs standard AST method (micro-broth dilution) to the sputum samples. We used 5 sputum samples with confirmed P.aeruginosa infection from adult CF patients.

Each sputum sample was divided into 4 parts (P1, P2, P3, P4).

P1 was resuspended with 1.0 ml PBS and plated to wells containing solid nutrient medium with antibiotics added at the mean concentration that can be achieved at the site of infection from 0-4 h and cultivated for 24 h at 37 C (Proposed Method).

P2 was directly plated (without being resuspended) to wells containing solid nutrient medium with antibiotics added at the mean concentration that can be achieved at the site of infection from 0-4h and cultivated for 24 h at 37 C.

P3—will proceed with a routine microbroth-dilution method. Antibiotic efficacy against P. aeruginosa were categorized as Susceptible or Resistant according to Clinical and Laboratory Standards Institute recommendations.

Narrow spectrum antibiotics used: Aztreonam, Vancomycin, Colistin, Cephalexin,

Next we tested the efficacy of antibiotics selected as effective within P1-P3 and added them to the remaining part of biosamples (marked as P4). The number of viable P.aeruginosa CFU/mL were evaluated. The decrease of P.aeruginosa CFU/mL by (i) 4log10 or (ii) total elimination were suggested effective. The results are shown in Table 18.

TABLE 18
Total number of narrow spectrum antibiotics suggested as effective
against P. aeruginosa determined by different methods.

| | Total number of narrow spectrum antibiotics | | | | | |
|---|---|---|---|---|---|---|
| | suggested as effective against P. aeruginosa | | | | | |
| | | | Direct plating without | | | |
| | Proposed method | | being resuspended | | Standard AST | |
| | Decrease by | | Decrease | | Decrease | |
| Biosample | 4log10 | Total | by 4log10 | Total | by 4log10 | Total |
| # | CFU/mL | elimination | CFU/mL | elimination | CFU/mL | elimination |
| 1 | 2 | 1 | 2 | 1 | 0 | 0 |
| 2 | 0 | 0 | 0 | 0 | 0 | 0 |
| 3 | 0 | 0 | 0 | 0 | 0 | 0 |
| 4 | 2 | 2 | 1 | 1 | 0 | 0 |
| 5 | 3 | 1 | 2 | 1 | 1 | 0 |
| Total | 7 | 4 | 5 | 3 | 1 | 0 |

As it is seen, unexpectedly, Proposed Method enabled higher rate of narrow spectrum antibiotics selection compared with the method when biosample was directly plated to the medium and compared with the standard AST. Selection of narrow spectrum antibiotics is critical for antibiotic stewardship to reduce antibiotic resistance spread. Moreover, in many cases narrow spectrum antibiotics selected with Proposed method were able more frequently to completely eliminate P. aeruginosa, while other methods were not.

### Example 18. Use of the Described Method to Develop an Antibiotic Regimen to Indirectly Eradicate Microorganisms of Interest

We used the method described above to select antibiotics indirectly active against Pseudomonas aeruginosa (Primary Pathogen) from antibiotics that directly are not effective against P. aeruginosa. We used biosamples form the experiment above. Two antibiotics Vancomycin and Cephalexin known to be not active (according to standard micro-broth dilution method) against Pseudomonas aeruginosa were added to the sputum samples with known Pseudomonas aeruginosa. We evaluated how these antibiotics indirectly active against Pseudomonas aeruginosa could be effective in eliminating Pseudomonas aeruginosa in mixed biofilms (table 19).

TABLE 19
The efficacy of antibiotics indirectly
active against bacteria in the samples

| | Decrease by | Total |
|---|---|---|
| Biosample # | 4log10 CFU/mL | eradication |
| 1 | + | + |
| 2 | − | − |
| 3 | − | − |
| 4 | + | − |
| 5 | + | + |
| 6 | + | − |
| 7 | − | − |
| 8 | + | + |
| 9 | − | − |
| 10 | − | − |
| Total number of P. aeruginosa | 5 | 3 |
| eradications | | |

As it is seen, Proposed method enabled selection of antibiotics that were indirectly active against Pseudomonas aeruginosa and were capable to eradicate P. aeruginosa, without of direct targeting of this pathogen, while Standard AST (microbroth dilution) were not able to identify such drugs as effective.

### Example 19. Method of Antibiotic Selection Solely against Primary Pathogens and not against all Microorganisms that give a Visible Growth on the Medium

Four sputum samples from patients with lung infections were used. Bacterial content of the top-4 primary microorganisms identified in the samples are listed in Table 20 by MALDI-TOF. Pure bacterial culture of each microorganisms was obtained by routine microbiological methods. The results are shown in Table 20.

TABLE 20
Compositions of bacteria mixes

| | Bacterial mix #1 | Bacterial mix #2 | Bacterial mix #3 | Bacterial mix #4 |
|---|---|---|---|---|
| Primary | P. aeruginosa | P. aeruginosa | P. aeruginosa | P. aeruginosa |
| pathogen | | | | |
| Microbial | E. coli | R. dentocariosa | S. aureus | E. coli |
| Modulators | S. anginosus | A. ursingii | B. thailandensis | S. haemolyticus |
| | B. sonorensis | B. pumilus | | |

Minimal inhibitory concentrations (MICs) against the primary pathogen were determined by micro-broth dilution method against Meropenem, Cefepime, Ceftazidime, Amikacin.

If the MIC of certain antibiotic against PP was over 16 μg/mL, PP was suggested as resistant. In this case this antibiotic at MIC of 16 ug/mL was used in the following studies. PP alone, or Microbial Modulators (MM) alone or the original sputum were plated on the wells of 24 well plate filled with nutrient Columbia agar supplemented with erythrocytes 5% and supplemented with antibiotics taken as MIC determined against PP, but not more than 16 μg/mL and incubated 24 h at 37 C. The results are shown in Table 21

TABLE 21
Results of individual and complex sensitivity to antibiotics of bacterial mixes

| | | | | | | | | | | Bacterial set #3 | | | | Bacterial set #4 | | | |
|---|---|---|---|---|---|---|---|---|---|---|---|---|---|---|---|---|---|
| | | | | | | | | | | Ceftazidime (2 μg/mL) | | | | Amikacin (1 μg/mL) | | | |
| Bacterial set #1 | | | | | Bacterial set #2 | | | | | | | | Presence | | | | Presence |
| Meropenem (16 μg/mL) | | | | | Cefepime (16 μg/mL) | | | | | | | | of PP | | | | of PP |
| | | | | Mix | | | | | Mix | | | | in Mix | | | | in Mix |
| PP | BM | | | PP + MM | PP | BM | | | PP + MM | PP | BM | | PP + MM | PP | BM | | PP + MM |
| PA | SA | BS | EC | MIX | PA | RD | AU | BP | MIX | PA | SA | BT | MIX | PA | EC | SH | MIX |
| + | − | − | + | − | + | − | + | + | − | + | + | + | − | + | − | + | − |

PP—primary pathogen,
BM—Bacteria modulators,
MIX—PP +BM
Absence of microbial growth, means antibiotic is effective (marked as “−”)
Presence of microbial growth, means antibiotic is ineffective (marked as “+”)

As it seen from the table, data on antibiotic efficacy against PP were different when bacteria were cultured alone or with MM. As it is seen for Meropenem and Cefepime in Bacterial sets #1 and 2 respectively, although the PP was resistant to these antiboitics when grown as a monobacterial culture, when cultured together with MM in the form of Mixed culture , surprisingly became sensitive to it and did not gave growth on the media.

An opposite effect was noted in Bacterial Mixes #3,4, Ceftazidime and Amikacin were not active against when P.aeruginosa (Primary Pathogen) when it was cultivated alone, but once it was grown together with MM, the same antibiotics were able to eradicate this PP; however, other bacteria form the microbial mix continued to grow.

As it is seen, unexpectedly, proposed method enables selection of effective antibiotics even in mixed probes with visible growth on the medium.

### Example 20. Role of Use of Dilution on the Timing of the Appearance of Bacterial Growth

We used biosamples (3 sputum, 3 broncho-alveolar lavage, 3 throat swabs, 3 scapings form diabetic wound ulcer) isolated from patients with pneumonia (community acquired, hospital acquired, ventilator-associated), cystic fibrosis, patients prior to lung transplantation, chronic obstructive disease.

Biosamples were plated to the solid nutrient media Columbia and Pepted Meat agar, LB agar, Columbia agar all supplemented with erythrocytes 5%.

Group 1—plated directly

Group 2—plated following the vortex 60s

Group 3—plated after the adding of 1 ml PBS to 1 ml of biosample or per 1 swab

Group 4—plated after the adding of 1 ml PBS to 1 ml of biosample or per 1 swab and vortex 60 s

Probes were incubated at 37 C and hourly analyzed for the presence of bacterial growth by visual monitoring of the appearance of bacterial growth in each probe, looking at the plate at the angle of 45 degrees with a naked eye. The results are shown in Table 22.

TABLE 22
Effects of different media and dilution on the
time of the first signs of bacterial growth

| | Time of the first signs of bacterial growth | | | | | | | | | | | |
|---|---|---|---|---|---|---|---|---|---|---|---|---|
| | Group 1 | | | Group 2 | | | Group 3 | | | Group 4 | | |
| Probe | MM | LB | CA | MM | LB | CA | MM | LB | CA | MM | LB | CA |
| Sputum | 3.5 | 4.5 | 4.5 | 3.5 | 4 | 4.5 | 3 | 3 | 3 | 3 | 3 | 3 |
| BAL | 3.5 | 5 | 5 | 4 | 4.5 | 4.5 | 2.5 | 3.5 | 3.5 | 2 | 3 | 3 |
| Throat swab | 4 | 4.5 | 4.5 | 4 | 4 | 4 | 4 | 3 | 3.5 | 3.5 | 3 | 3.5 |
| Scaping | 4.5 | 5 | 5 | 4 | 4.5 | 4.5 | 3 | 3.5 | 3.5 | 3 | 3 | 3 |

MM—mixed medium (Columbia agar + Pepted Meat agar),
LB—Luria Broth agar,
CA—Columbia agar

Next, we analyzed, how accurate are the results obtained by monitoring early signs of microbial growth in terms for their progression to growth during the first 24 h. We were particularly interested in possible very major errors (VME), defined as when according to the test system, antibiotic was effective (no bacterial growth) at the first timepoint of 2.5-5 h, but then, it progressed to the appearance of growth during the subsequent cultivation for 24 h (FIG. 15).

As it is seen, unexpectedly, use of solvents and/or vortex prior to probe sampling significantly increased the speed bacterial growth and accuracy of appearance and progression bacterial growth.

### Example 21. Use of Proposed Method within Less than 4 Hours for the Selection of Antibiotics Effective against Persisters and/or Preventing Antibiotic-Induced Formation of Persisters

Pathological material (sputum) from patients with lung diseases was plated into wells of 24-well plates filled with a nutrient medium supplemented with erythrocytes 5%, to which antibiotics were added taken at different concentrations.

Group #1—antibiotics were taken at concentrations close to a maximum (peak) concentration achievable at the site of infection Cmax

Group #2—antibiotics were taken at mean concentrations achievable at the site of infection from 0 to 4 h post antibiotic administration (C0-4 h)

Algorithm #1. Lab technician, analyzed the appearance of bacterial growth by comparing the signs of bacterial growth in each well supplemented with antibiotic(s) at certain timepoint, with the signs of bacterial growth in the same well at previous time point(s).

Algorithm #2. Lab technician analyzed the appearance of bacterial growth by comparing the signs of bacterial growth in each well supplemented with antibiotic(s) with bacterial growth in antibiotic-free control

Algorithm #3—Standard antibiotic susceptibility study using the microbroth dilution method. The results are shown in Table 23.

TABLE 23
Comparison of different data analysis algorithms on the speed of antibiotic selection

| | Algorithm | | | | | | | | | |
|---|---|---|---|---|---|---|---|---|---|---|
| | #1 | | | | #2 | | | | Microbroth | |
| | Time to | | Time to | | Time to | | Time to | | dilution method | |
| | results of | | results of | | results of | | results of | | Time to | |
| | antibiotic | | antibiotic | | antibiotic | | antibiotic | | results of | |
| | selection | | selection | | selection | | selection | | antibiotic | |
| | (h) | ATB | (h) | ATB | (h) | ATB | (h) | ATB | selection | ATB |
| Antibiotic | Cₘₐₓ | efficacy | C_{0-4 h} | efficacy | Cₘₐₓ | efficacy | C_{0-4 h} | efficacy | (h) | efficacy |
| Vancomycin | 4.5 | − | 3 | + | 5 | − | 4.5 | + | 42 | − |
| Cefepime | 3 | − | 2.5 | − | 4 | − | 4 | − | 42 | + |
| Ceftazidine | 3.5 | + | 3 | + | 4.5 | + | 4 | + | 42 | + |
| Amikacin | 3 | − | 3 | − | 5 | − | 4 | − | 42 | − |

Atb—antibiotic;
“+”—presence of bacterial growth, antibiotic ineffective;
“−”—absence of bacterial growth, antibiotic effective

It is clearly seen that the use of antibiotics taken at the lower concentration (not maximum concentration achievable at the site of infection), allows to achieve quicker evaluation of antibiotic efficacy in mixed microbial communities, moreover, the spectrum of antibiotics suggested as effective differs between probes where antibiotics were added at Cmax vs C0-4h concentrations.

Next, we confirmed that antibiotics selected with proposed algorithm are effective. Adult C57BL/6 mice were rendered neutropenic by injecting cyclophosphamide subcutaneously 4 days before infection (150 mg/kg of body weight), 1 day before infection (100 mg/kg), and 1 day after infection (100 mg/kg). Mice were anesthetized with 2% isoflurane and orally instilled with biosample (stored at +4 C). Briefly, nares were blocked, and mice aspirated 50 μL of the biosample into the lungs while being held vertically for 60 s.

Group #1—Animals (n=8) were treated with vancomycin for 72 h—an antibiotic that was determined as effective according to (1) microbroth dilution, and (2) proposed method with antibiotics added to the medium at the concentration Cmax, but was suggested as ineffective according to proposed method with antibiotics added to the medium at the concentration C04 h.

Group #2—Animals (n=8) were treated with Cefepime for 72 h—an antibiotic that was determined as effective according to microbroth dilution, but were suggested as ineffective according to proposed method with antibiotics added to the medium at the concentrations Cmax and C0-4h.

Lungs and liver were collected and homogenized in 1 mL phosphate-buffered saline and then serial tenfold dilutions of tissue homogenates were plated on Columbia agar with 5% sheep blood supplemented with 100 ug/mL Vancomycin or Cefepime. The results are shown in Table 24.

TABLE 24
Presence of microbial growth in the organs and blood of animals treated
with antibiotics selected as effective with different algorithms.

| | Number of animals with positive microbial growth on the | | |
|---|---|---|---|
| | agar with antibiotics/total number of animals per group | | |
| Antibiotic | Blood | Lungs | Liver |
| Vancomycin | 6/8 | 8/8 | 5/8 |
| Cefepime | 4/8 | 3/8 | 2/8 |

As it is seen from these results, antibiotics selected based on a proposed method, when antibiotics are taken at the concentration that is lower (C0-4 h) than maximum achievable at the site of infection and are selected not only against primary pathogens, bur also against microbial modulators enable more precise selection of antibiotics that are active against persisters or prevent persisters formation. The use of antibiotics taken at the concentration Cmax underestimates the diversity of antibiotics (including narrow-spectrum) that are actually effective, while adding of antibiotics to the test system at lower concentrations provides more accurate results.

Surprisingly, we found that the adding of antibiotics at concentrations lower than maximum achievable at the site of infection—C0-4 h, enables to select antibiotics quicker compared with the test systems when antibiotics were added at Cmax concentration. Moreover, the use Algorithm #1 when the signs of bacterial growth in each well supplemented with antibiotic(s) at certain timepoint are compared with the signs of bacterial growth in the same well at previous time point, provides faster data analysis.

### Example 22. Evaluation of the Effects of Bacterial Modulators on Primary Pathogens from the Biosamples of the Diseases with Less Bacterial Diversity Compared with Cystic Fibrosis

We used biosamples (sputum, throat swabs, scapings form diabetic wound ulcer, swabs from burns, urine, synovial fluid) isolated from patients with pneumonia, chronic obstructive pulmonary disease, diabetic wound, burns, arthritis, cystitis.

Biosamples were processed as the following: after the adding of 1 ml PBS to 1 ml of biosample or per 1 swab and vortex 60 s and incubated at 37 C for 4 h.

Microbial diversity was assessed by culture-based techniques. To confirm that identified microogramisms are Microbial Modulators we isolated Primary Pathogen from the media with and without of Microbial Modulators and monitored the expression of antibiotic resistance profile using transcriptome analysis with (Illumina HiSeq4000) to evaluate have Microbial Modulators affected the expression of antibiotic resistance genes of Primary Pathogen.

The expression of antibiotic resistance genes of primary pathogens was assessed against the Uniprot and NCBI protein databases (Table 25).

TABLE 25
Evaluation of the effects of bacterial modulators on primary pathogens

| | Predominant Pathogen | | | | | |
|---|---|---|---|---|---|---|
| | | Chronic | | | | |
| | | obstructive | | | | |
| | | pulmonary | Diabetic | | | |
| | Pneumonia | disease | wound | Burns | Arthritis | |
| | Streptococcus | Haemophilus | Enterococcus | Staphylococcus | Staphylococcus | Cystitis |
| | pneumoniae | influenzae | spp | aureus | aureus | E. coli |
| Total number | 3 | 7 | 5 | 4 | 2 | 2 |
| of potential MM | | | | | | |
| Relative | 100% | 100% | 100% | 100% | 100% | 100% |
| number of | | | | | | |
| overexpressed | | | | | | |
| ARG in PP | | | | | | |
| cultured alone | | | | | | |
| Number of | 46% | 552% | 217% | 33% | 190% | 824% |
| overexpressed | | | | | | |
| ARG in PP | | | | | | |
| cultured | | | | | | |
| together with MM | | | | | | |

These results clearly show that the probes even with a small bacterial diversity can have MMs that modulate the expression of ARG in PP.

### Example 23. Test Systems with Individual Antibiotic Composition for the Selection of Effective Concentration-Dependent Antibiotics for Therapy in Persons with Altered Pharmacokinetics Children, Persons with Hypersensitivity to Antimicrobial Drugs

We next unexpectedly discovered that by using a proposed method of combined cultivation of PP and MM on the media supplemented with different concentrations of concentration-dependent antibiotics we are able to develop more safe and effective antibiotic regimen and select more effective antibiotics compared to the regular methods based on the standard guidelines.

We used sputum from patients with different pulmonary infections. Pure bacterial culture of each microorganisms was obtained by routine microbiological methods, bacterial identification was done by MALDI-TOF analysis, microbial diversity of biosample was done by metagenomic analysis (16S RNA sequencing).

Primary pathogen was selected as a leading pathogen in each infection case, based on consultation with an independent doctors who have received a description of the clinical picture, microbiology and microbiology analyses. Sensitivity of PP to antibiotics was evaluated using a standard serial microbroth-dilution method. The results are shown in Table 26.

TABLE 26
Baseline characteristics of Primary Pathogens

| | |
|---|---|
| Identified Primary | Tobramycin* |

| Biosample | Pathogen | MIC μg/mL | Sensitivity |
|---|---|---|---|
| 1 | P. aeruginosa | 2 | S |
| 2 | P. aeruginosa | 8 | I |
| 3 | P. aeruginosa | 8 | I |

*Tobramycin is a Concentration-dependent antibiotic
Next to antibiotic's MIC in μg/mL the susceptibility interpretation: S (sensitive), I (intermediate) or R (resistant) is added established by the Clinical and Laboratory Standards Institute (CLSI).

Next, biosamples were plated to the wells of 48 well plate filled with Columbia +Pept Meat Agar supplemented with tobramycin taken at different concentrations (we have taken MIC as equal to Cmax) and presence of PP growth was evaluated in each well in 24 h of growth. The results are shown in Table 27.

TABLE 27
Presence of bacterial growth in the wells
with different Tobramycin concentrations

| | Presence of bacterial growth in the wells | | | | | | |
|---|---|---|---|---|---|---|---|
| Biosample# | with different Tobramycin concentrations | | | | | | |
| (PP is P. | Cₘₐₓ × | Cₘₐₓ × | | ½ | ¼ | 1/10 | 1/100 |
| aeruginosa) | 10 | 2 | Cₘₐₓ | Cₘₐₓ | Cₘₐₓ | Cₘₐₓ | Cₘₐₓ |
| 1 | − | − | − | − | − | − | + |
| 2 | − | − | − | + | + | + | + |
| 3 | − | − | − | − | − | + | + |

“+”—presence of bacterial growth, antibiotic is ineffective
“−”—absence of bacterial growth, antibiotic is effective

Next, using the same biosamples we infected mice modulating pneumonia. For that, adult C57BL/6 mice were rendered neutropenic by injecting cyclophosphamide subcutaneously 4 days before infection (150 mg/kg of body weight), 1 day before infection (100 mg/kg), and 1 day after infection (100 mg/kg). Mice were anesthetized with 2% isoflurane and orally instilled with biosample disluted in PBS. Briefly, nares were blocked, and mice aspirated 50 μL of the biosample into the lungs while being held vertically for 60 s. MIX was taken as a Cmax.

30 animals were used (n=10 per group) and were treated with different concentrations of tobramycin according to the results obtained by proposed method from 3 different biosamples. Five animals from each group—received Tobramycin at concentration suggested as the minimally effective, and 5 animals from each group received tobramycin at concentration that was suggested as the first ineffective concentration with a proposed method. The results are shown in Table 28.

TABLE 28
Detailed description of the animal groups

| | | | Treated with | Treated with |
|---|---|---|---|---|
| | | | tobramycin taken | tobramycin taken |
| | | | at concentration | at concentration |
| | # of | Infected | suggested as | suggested as |
| | animals | with | the lowest | the highest |
| Group | per group | biosample | EFFECTIVE | INEFFECTIVE |
| 1a | 5 | #1 | Cmax 1/10 | N/A |
| 1b | 5 | #1 | N/A | Cmax 1/100 |
| 2a | 5 | #2 | Cmax 1/1 | N/A |
| 2b | 5 | #2 | N/A | Cmax 1/10 |
| 3a | 5 | #3 | Cmax 1/4 | N/A |
| 3b | 5 | #3 | N/A | Cmax 1/10 |

The efficacy or inefficacy of treatment with tobramycin taken at different concentrations is presented in the table below. Efficacy was evaluated by the decrease of PP count by 3log10 over 48 hours. The results are shown in Table 29.

TABLE 29
In vivo confirmation of antibiotic efficacy

| | | | Treated with | Treated with | Number of |
|---|---|---|---|---|---|
| | | | tobramycin | tobramycin | animals to |
| | | | taken at | taken at | whom therapy |
| | | | concentration | concentration | was effective/ |
| | # of | Infected | of suggested | of suggested | number of |
| | animals | with | as the lowest | as the highest | animals per |
| Group | per group | biosample | EFFECTIVE | INEFFECTIVE | group |
| 1a | 5 | #1 | Cmax 1/10 | N/A | 5/5 |
| 1b | 5 | #1 | N/A | Cmax 1/100 | 0/5 |
| 2a | 5 | #2 | Cmax 1/1 | N/A | 5/5 |
| 2b | 5 | #2 | N/A | Cmax 1/10 | 0/5 |
| 3a | 5 | #3 | Cmax 1/4 | N/A | 5/5 |
| 3b | 5 | #3 | N/A | Cmax 1/10 | 0/5 |

Thus, the proposed method made it possible to select an effective therapy using a lower dosage of antimicrobial drugs, in contrast to the standard method, which would suggest using the drug based on its MIC concentration. It is important for people with underlying diseases, altered pharmacodynamic parameters, and children. In addition, the proposed method—with 100% accuracy allows you to determine the concentration of the antibiotic and the therapeutic course, which is not effective.

### Example 24. Test Systems with Individual Antibiotic Composition for the Selection of Effective Time-Dependent Antibiotics for Therapy in Persons with Altered Pharmacokinetics, Children, Persons with Hypersensitivity to Antimicrobial Drugs

We next unexpectedly discovered that by using a proposed method of combined cultivation of PP and MM on the media supplemented with different concentrations of time-dependent antibiotics we are able to develop more safe and effective antibiotic regimen and select more effective antibiotics compared to the regular methods based on the standard guidelines.

We used sputum from patients with different pulmonary infections. Pure bacterial culture of each microorganisms was obtained by routine microbiological methods, bacterial identification was done by MALDI-TOF analysis, microbial diversity of biosample was done by metagenomic analysis (16S RNA sequencing).

Primary pathogen was selected as a leading pathogen in each infection case, based on consultation with an independent doctors who have received a description of the clinical picture, microbiology and microbiology analyses. Sensitivity of PP to antibiotics was evaluated using a standard serial microbroth-dilution method. The results are shown in Table 30.

TABLE 30
Baseline characteristics of S. aureus

| | |
|---|---|
| Identified Primary | Amoxicillin* |

| Biosample | Pathogen | MIC μg/mL | Sensitivity |
|---|---|---|---|
| 1 | S. aureus | 0.25 | S |
| 2 | S. aureus | 4 | I |
| 3 | S. aureus | 2 | S |

*Amoxicillin is a time-dependent antibiotic
Next to antibiotic's MIC in μg/mL the susceptibility interpretation: S (sensitive), I (intermediate) or R (resistant) is added established by the Clinical and Laboratory Standards Institute (CLSI).

Next, biosamples were plated to the wells of 48 well plate filled with Columbia+Pept Meat Agar supplemented with Amoxicillin. We modulated a QID, BID and TID administration of amoxicillin on PP+MM within the biosample. The following assumptions were made. The results are shown in Table 31.

TABLE 31
Relationships between concentration of antibiotic
used tets system and in vivo administration regimen

| Concentration | | Equivalent to |
|---|---|---|
| of antibiotic | | administration |
| used tets system | Comment | regimen |
| C0-6 h | Concentration of antibiotic | TID |
| | taken at the mean | |
| | concentration of drug from | |
| | 0-6 hours | |
| C0-12 h | Concentration of antibiotic | BID |
| | taken at the mean | |
| | concentration of drug from | |
| | 0-12 hours | |
| C0-24 h | Concentration of antibiotic | QID |
| | taken at the mean | |
| | concentration of drug from | |
| | 0-24 hours | |

Next we evaluated Presence of bacterial growth in the wells supplemented with amoxicillin. The results are shown in Table 32.

TABLE 32
Presence of bacterial growth in the
wells supplemented with amoxicillin

| | Presence of bacterial growth in the | | |
|---|---|---|---|
| Biosample # | wells supplemented with amoxicillin | | |
| (PP is S. aureus) | C0-6 h | C0-12 h | C0-24 h |
| 1 | − | − | − |
| 2 | − | + | + |
| 3 | − | − | + |

“+” - presence of bacterial growth, antibiotic is ineffective
“−”- absence of bacterial growth, antibiotic is effective

25 animals were used (n=5 per group) and were treated with different concentrations of Amoxicillin according to the results obtained by proposed method from 3 different biosamples. Five animals from each group—received Amoxicillin at the schedule as evaluated with proposed method (equivalent to TID for C0-6 h, BID for C0-12 h or QID C-24 h), and 5 animals from groups #2 and #3 received Amoxicillin at the regimen that was suggested as the first ineffective. The results are shown in Table 33.

TABLE 33
Detailed description of the animal groups

| | | | Treated with | Treated with |
|---|---|---|---|---|
| | | | Amoxicillin | Amoxicillin |
| | | | taken at regimen | taken at regimen |
| | # of | Infected | suggested as | of suggested as |
| | animals | with | the lowest | the highest |
| Group | per group | biosample | EFFECTIVE | INEFFECTIVE |
| 1a | 5 | #1 | QID | N/A |
| 2a | 5 | #2 | TID | N/A |
| 2b | 5 | #2 | N/A | BID |
| 2a | 5 | #3 | BID | N/A |
| 2b | 5 | #3 | N/A | QID |

The efficacy or inefficacy of such a treatment is presented in the table below. Efficacy was evaluated by the decrease of PP count by 3log10 over 48 hours. The results are shown in Table 34.

TABLE 34
In vivo confirmation of antibiotic efficacy

| | | | Treated with | Treated with | Number of |
|---|---|---|---|---|---|
| | | | tobramycin | tobramycin | animals to |
| | | | taken at | taken at | whom therapy |
| | | | concentration | concentration | was effective/ |
| | # of | Infected | of suggested as | of suggested as | number of |
| | animals | with | the lowest | the highest | animals per |
| Group | per group | biosample | EFFECTIVE | INEFFECTIVE | group |
| 1a | 5 | #1 | QID | N/A | 5/5 |
| 2a | 5 | #2 | TID | N/A | 5/5 |
| 2b | 5 | #2 | N/A | BID | 0/5 |
| 3a | 5 | #3 | BID | N/A | 5/5 |
| 3b | 5 | #3 | N/A | QID | 0/5 |

Thus, the proposed method made it possible to select an effective regimen for drug administration, including the use of more rare and optimized dosage regimens for antimicrobial drugs. It is important for people with underlying diseases, altered pharmacodynamic parameters, and children. In addition, the proposed method—with 100% accuracy, allows you to determine a therapeutic course that is not effective.

### Example 25. Evaluation of Probability Model for the Proposed Diagnostic Method

We next tried to select antibiotics based on building a probability model. Antibiotic efficacy was evaluated based on a novel parameters:
ABEPPMM100—antibiotic concentration that is required for the elimination of 100% “complex” PP+MM.
ABEPPMM99—antibiotic concentration that is required for the elimination of 99% “complex” PP+MM.

ABEPPMM90—antibiotic concentration that is required for the elimination of 90% “complex” PP+MM.

ABEPPMM50—antibiotic concentration that is required for the elimination of 50% “complex” PP+MM.

ABEPP100—antibiotic concentration that affecting on “complex” PP+MM is required for the elimination of 100% PP.

ABEPP99—antibiotic concentration that affecting on “complex” PP+MM is required for the elimination of 99% PP.

ABEPP90—antibiotic concentration that affecting on “complex” PP+MM is required for the elimination of 90% PP.

ABEPP50—antibiotic concentration that affecting on “complex” PP+MM is required for the elimination of 50% PP.

We assumed that in cases where infections are caused by multidrug-resistant microorganisms and it is impossible to select an effective antibiotic with the achievement of ABEPPMM100 or ABEPP100 value (i.e. due to the individual characteristics of the macroorganism (concomitant diseases, features of the antibiotic pharmacokinetics)), it is necessary to select antibiotics with the highest probability of being therapeutically effective.

We used sputum from children with pneumonia, with S.aureus as confirmed PP. Patient 1—Boy 10 y.o.
Patient 2—Boy 5 y.o.
Patient 3—Girl 7 y.o.

Biosamples were plated on the wells of a multi-well plate filled with Columbia agar supplemented with antibiotic (non-limiting examples of levofloxacin, cefepime) taken at different concentrations achievable at the site of infection and PP and MM presence was analyzed after 24 h of growth.

Cmax×2=maximum peak antibiotic concentration achievable at the site of infection×2.
C0-1h—mean concentration of antibiotic achievable at the site of infection from 0 to 1 hour post administration
C0-2h—mean concentration of antibiotic achievable at the site of infection from 0 to 2 hour post administration
C0-3h—mean concentration of antibiotic achievable at the site of infection from 0 to 3 hour post administration
C1-3h—mean concentration of antibiotic achievable at the site of infection from 0 to 1 hour post administration
C0-4h—mean concentration of antibiotic achievable at the site of infection from 0 to 4 hour post administration
C2-3h—mean concentration of antibiotic achievable at the site of infection from 2 to 3 hour post administration
C4-6h—mean concentration of antibiotic achievable at the site of infection from 4 to 6 hour post administration
C0-6h—mean concentration of antibiotic achievable at the site of infection from 0 to 6 hour post administration
C6-8h—mean concentration of antibiotic achievable at the site of infection from 6 to 8 hour post administration
C0-8h—mean concentration of antibiotic achievable at the site of infection from 0 to 8hour post administration
C8-12h—mean concentration of antibiotic achievable at the site of infection from 8 to 12 hour post administration
C0-12h—mean concentration of antibiotic achievable at the site of infection from 0 to 12 hour post administration
C12-24h—mean concentration of antibiotic achievable at the site of infection from12 to 24 hour post administration
C0-24h—mean concentration of antibiotic achievable at the site of infection from 0 to 24 hour post administration

The results are shown in Tables 35 and 36.

TABLE 35
Evaluation of Levofloxacin efficacy

| | Degree of primary pathogen eradication | | | | | | | | | | | | | | |
|---|---|---|---|---|---|---|---|---|---|---|---|---|---|---|---|
| | Cₘₐₓ × 2 | | C0-1 h | | C0-2 h | | C2-3 h | | C0-3 h | | C3-4 h | | C0-4 h | | C4-6 h |
| Levofloxacin | 1 | 2 | 1 | 2 | 1 | 2 | 1 | 2 | 1 | 2 | 1 | 2 | 1 | 2 | 1 |
| Patient 1 | 99 | 100 | 99 | 100 | 99 | 99 | 90 | 90 | 90 | 90 | 99 | 99 | 100 | 100 | 100 |
| Patient 2 | X | X | X | X | X | x | X | X | 50 | 50 | 50 | 50 | 50 | 50 | 50 |
| Patient 3 | X | X | X | X | X | X | X | X | 50 | 50 | 50 | 50 | 0 | 50 | 0 |

| | Degree of primary pathogen eradication | | | | | | | | | | | | | | |
|---|---|---|---|---|---|---|---|---|---|---|---|---|---|---|---|
| | C4-6 h | C0-6 h | | C6-8 h | | C0-8 h | | C8-12 h | | C0-12 h | | C12-24 h | | C0-24 h | |
| Levofloxacin | 2 | 1 | 2 | 1 | 2 | 1 | 2 | 1 | 2 | 1 | 2 | 1 | 2 | 1 | 2 |
| Patient 1 | 100 | 100 | 100 | 90 | 90 | 50 | 50 | 50 | 50 | 50 | 50 | 50 | 50 | 0 | 0 |
| Patient 2 | 50 | 50 | 50 | 50 | 50 | 50 | 50 | 0 | 50 | 0 | 50 | 0 | 0 | 0 | 0 |
| Patient 3 | 0 | 0 | 0 | 0 | 0 | 0 | 0 | 0 | 0 | 0 | 0 | 0 | 0 | 0 | 0 |

1 = evaluation of ABEPPMM;
2 = evaluation of ABEPP;
0—antibiotic does not reach any efficacy within ABEPPMM-ABEPP50-100 range
X—antibiotic can not be prescribed in such a concentration due to individual particularities.

TABLE 36
Evaluation of Cefepime efficacy

| | Degree of primary pathogen eradication | | | | | | | | | | | | | | |
|---|---|---|---|---|---|---|---|---|---|---|---|---|---|---|---|
| | Cₘₐₓ × 2 | | C0-1 h | | C0-2 h | | C2-3 h | | C0-3 h | | C3-4 h | | C0-4 h | | C4-6 h |
| Cefepime | 1 | 2 | 1 | 2 | 1 | 2 | 1 | 2 | 1 | 2 | 1 | 2 | 1 | 2 | 1 |
| Patient1 | 100 | 100 | 100 | 100 | 100 | 100 | 100 | 100 | 100 | 100 | 100 | 100 | 100 | 100 | 100 |
| Patient2 | X | X | X | X | X | X | X | X | 90 | 90 | 50 | 50 | 50 | 50 | 50 |
| Patient3 | X | X | X | X | X | X | X | X | 90 | 90 | 90 | 90 | 90 | 90 | 90 |

| | Degree of primary pathogen eradication | | | | | | | | | | | | | | |
|---|---|---|---|---|---|---|---|---|---|---|---|---|---|---|---|
| | C4-6 h | C0-6 h | | C6-8 h | | C0-8 h | | C8-12 h | | C0-12 h | | C12-24 h | | C0-24 h | |
| Cefepime | 2 | 1 | 2 | 1 | 2 | 1 | 2 | 1 | 2 | 1 | 2 | 1 | 2 | 1 | 2 |
| Patient1 | 100 | 100 | 100 | 100 | 100 | 50 | 50 | 00 | 50 | 00 | 00 | 00 | 00 | 0 | 0 |
| Patient2 | 50 | 50 | 50 | 50 | 50 | 50 | 50 | 50 | 50 | 50 | 50 | 0 | 0 | 0 | 0 |
| Patient3 | 90 | 90 | 90 | 0 | 0 | 0 | 0 | 0 | 0 | 0 | 0 | 0 | 0 | 0 | 0 |

1 = evaluation of ABEPPMM;
2 = evaluation of ABEPP;
0—antibiotic does not reach any efficacy within ABEPPMM-ABEPP50-100 range
X—antibiotic can not be prescribed in such a concentration due to individual particularities.

As can be seen from the data presented in Table 1, Levofloxacin for patient #1 is most effective. At the same time, patients 2 and 3 (children with impaired liver function) cannot receive the maximum dose of the antibiotic.

To assess which antibiotic Levofloxacin or Cefepime is most effective for patients 2 and 3, we used a probabilistic model.

As can be seen, the antibiotic Levofloxacin works in both patients at higher concentrations. But—for patient #2, Levofloxacin at a concentration equivalent from C0-3h to C0-12h allows you to destroy 50% of the “complex” of PP+MM. And for patient-3, Levofloxacin eliminates 50% of the “complex” of PP+MM in the higher concentration range from C0-3h to C0-4h.

The calculation of the probability of the effectiveness of Levofloxacin was carried out according to the formula:

$$Probability of antibiotic effectiveness = \frac{\begin{matrix}Maximum antibiotic “ X ” concentration to reach ABEPPMM 50 - \\ Minimum antibiotic “ X ” concentration to reach ABEPPMM 50\end{matrix}}{\begin{matrix}Maximum antibiotic “ X ” concentration to reach ABEPPMM 50 \times \\ Probability coefficient 50\end{matrix}}$$

Thus for Patient #2 the probability of Levofloxacin effectiveness=(4.7 ug/mL [lung concentration of Levofloxacin after 3 h of administration]−3.7 [lung concentration of Levofloxacin after 12 h of administration]ug/mL)/4.7 ug/mL [lung concentration of Levofloxacin after 3 h of administration]×Probability coefficient50=0.212×Probability coefficient50

for Patient #3 the probability of Levofloxacin effectiveness=(4.7 ug/mL [lung Levofloxacin of Levofloxacin after 3 h of administration]−4 ug/mL [lung concentration of cefepime after 3 h of administration])/4.7 ug/mL [lung concentration of cefepime after 3 h of administration]×Probability coefficient50=0.15×Probability coefficient50

Thus, the proposed method allows to evaluate that Levofloxacin will most likely be more effective for patient 2 compared to the effectiveness of the same antibiotic in patient 3.

The probability of Cefepime effectiveness for patient #2=(23 ug/mL [lung concentration of cefepime after 8 h of administration]−8 ug/mL [lung concentration of cefepime after 12 h of administration] ABEPPMM50)/23 [lung concentration of cefepime after 8 h of administration] ug/mL ×Probability coefficient50 =0.65×Probability coefficient50

for Patient #3 the probability of Cefepime effectiveness=(23 ug/mL [lung concentration of cefepime after 8 h of administration]−12 ug/mL [lung concentration of cefepime after 6 h of administration] ABEPPMM50)/23 ug/mL×Probability coefficient90 =0.47×Probability coefficient90

Thus, the proposed method allows to evaluate that Cefepime will most likely be more effective for patient 3 compared to the effectiveness of the same antibiotic in patient 2.

These data clearly show that the proposed method allows with varying probability to determine the effectiveness of antibiotics in individuals including those with metabolic characteristics and special groups of patients. Moreover, probabilities for any of ABEPPMM or ABEPP can be achieved with different probability.

Unexpectedly, we found that the selection of effective antimicrobial drugs in the proposed research method, when pathological material is inoculated on a nutrient medium with antibiotics taken at different concentrations, allows selecting effective antibiotics in different concentrations and determining the concentration of which antibiotics can be applied in less concentration, in less concentration persons with impacted excretion. as a result—less toxicity and less number of side effects and complications

### Example 26. Use of μ-Metal and Different Agar Stings to Stimulate Bacterial Growth

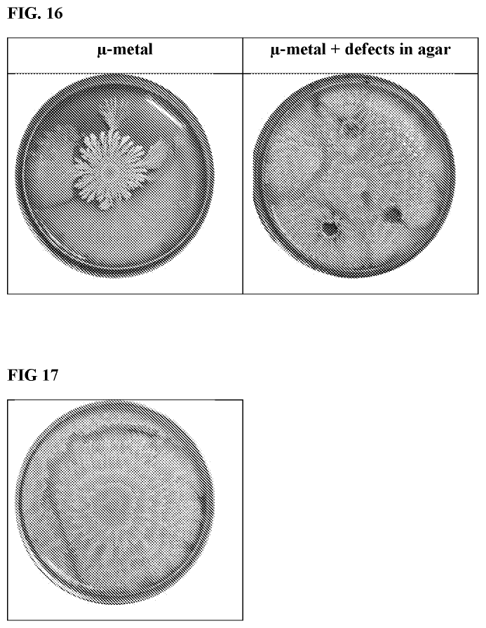

We used Mu-metal to regulate bacterial growth an memory. For that, we plated 10 uL overnight Bacillus pumilus VT 1200 on the surface of and mixed Columbia and Pepted Meat agar (unaltered or with small 5 mm in diameter round defects)on 90 mm Petri dishes, placed in Mu-metal boxes and cultivated for 24 h at 37 C (FIG. 16).

It is seen that the growth of bacteria in altered geomagnetic field on the media with alteration of its surface (i.e. electromagnetic surfaces) stimulated microbial growth diverse “wave-like” bacterial growth. We than plated bacteria from the plate “μ-metal+defects in agar” on unaltered agar and controlled bacterial growth for the next 24 h at 37 C (FIG. 17).

We unexpectedly discovered that stimulation of microbial growth is saved throughout multiple cell generations that can be used in biotechnology.

### Example 27. Use of μ-Metal Test Systems to Monitor Health State

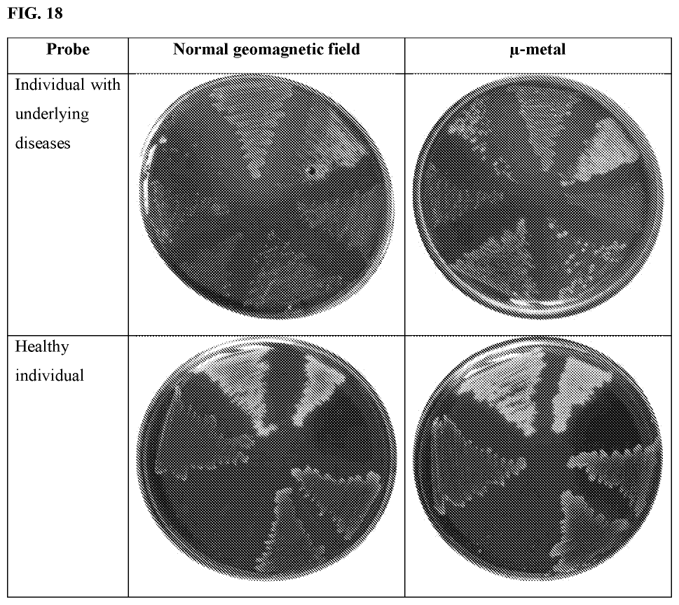

Saliva of healthy individual and subject with underlying diseases (migraine) was used and random 7 bacterial strains (as a pure culture or bacterial mix) were isolated. 10 uL overnight culture of each of them were plated on the surface of mixed Columbia and Pepted Meat agar supplemented with 10% erythrocytes on 90 mm Petri dishes, placed in Mu-metal boxes and cultivated for 24 h at 37 C. The results are shown in FIG. 18.

It is seen that the growth of bacteria from biosample of individual with certain diseases, led to the differential microbial growth in altered geomagnetic field, compared with bacteria from microbiota of healthy individual that were not changed following the change of geomagnetic field.

### Example 28. Use of Light to Increase Productivity of Cells in Biomanufacturing

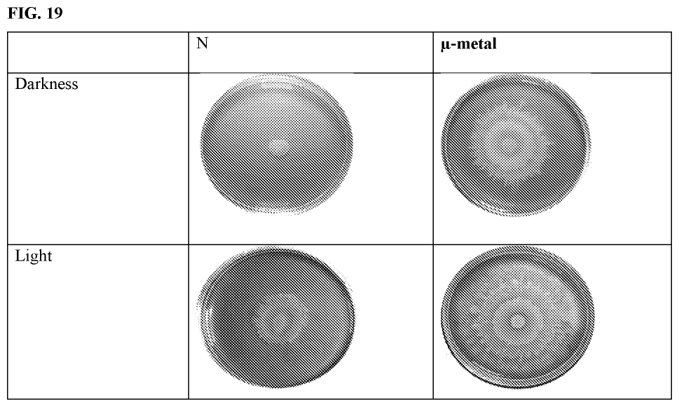

B.pumilus VT1200 were cultured on the agar of 90 mm Petri dishes and cultivated in the light environment for 48 h at 37 C. The results are shown in FIG. 19.

We also analyzed the role of light on cells productivity and biomass when cells were cultured in suspension culture (Table 37).

TABLE 37
Role of light and dark on potentiation of
microbial growth in altered magnetic field

| | N (CFU log10/mL/ | μ-metal (CFU log10/mL)/ |
|---|---|---|
| Conditions | OD600% to control) | OD600% to control) |
| Darkness | 8.8 ± 0.3/100% | 9.7 ± 0.4/159% |
| Light | 9.5 ± 0.3/149% | 10.9 ± 0.4/244% |

As it can be seen, the growth under the light environment facilitates cell growth and synthetic activity and thus can be used in biotechnology to increase productivity.

### Example 29. Algorithm for Antibiotic Selection

Each biosample is directly plated to the solid nutrient media inside the wells of each multi-well plate. Nutrient media in each well is supplemented with different antibiotics or combinations of these antibiotics taken at concentrations from Cmax, C1/2max, C1/4max, C1/10max, C1/50max, C1/100max, C 1/1000 max. After the cultivation of PP, MM or their mix, the antibiotics that prevent the growth of these PP, MM or their mix are suggested to have the highest probability to be effective determined by which “X” value in “C 1/x max” equation is the highest.

1. The product wherein automatic and/or semiautomatic station for the preparation of test-systems of different constructions, and/or test-systems and/or Bio adjustable Tetz incubators, and/or CO2 incubators and/or bioreactors and/or incubator-warm rooms of different construction for cultivation of microorganisms and/or cell cultures with and/or nutrient media, and/or media additives and/or supplements, differs in that enables individually to prepare media with programmed composition in relation to the object of study and/or regulate, control interaction of microorganisms and eucaryotic cells with physical, chemical, biological environmental factors, and/or alter growth rate of different not-yet culturable bacteria, and/or fungi within mixed microbial communities.

2. The methods for preparation of test-systems of different construction and/or cultivation of microorganisms with the use of nutrient media, media additives and supplements, methods of their usage, differs in that enables individually to prepare media with programmed composition in relation to the object of study and/or regulate, control interaction of microorganisms and eucaryotic cells with physical, chemical, biological environmental factors, and/or alter growth rate of different with a non-limiting examples not-yet culturable bacteria, fungi within mixed microbial communities, alter and/or enhance microbial and/or eukaryotic cells growth and/or synthetic activity, and/or determine the effect of antimicrobial drugs on them to select effective and/or ineffective antimicrobial agents and/or combinations thereof including those with as narrow as possible spectrum of action and/or with the highest safety profile and/or cheapest and/or with certain route of administration active against (1) drug resistant microorganisms and/or (2) the existing persisters or persisters that can be formed in the course of antibiotic therapy of the infection and/or sporeforming bacteria, (3) recurrent infections done by (3) simultaneous analysis of combinational effect of antimicrobial agents on Primary Pathogen (PP) and Microorganisms Modulators (MM), as well as devices and methods of accounting for the results of these actions, wherein said sample comprising at least one of fluid, water, food, beverage, biofluid, tissue, microbial probes, a pharmaceutical preparation, a mammalian sourced tissue and liquids, air, soil, surface, swabs, and any combination thereof for the use in biopharmaceutical manufacturing, medicine, veterinary, agriculture, bioengineering, drug development and discovery, ecology, space exploration, space technology, persons with altered metabolic parameters that alters the pharmacokinetics and pharmacodynamics of drugs and/or children in particular

3. The product of claim 1, wherein automatic or semiautomatic station/complex for the preparation of test-systems of different constructions with individual programmed properties, set of antimicrobial compounds and different supplements in variable concentrations and combinations, marking all the details and properties of the final product and creating its electronic passport, preparing the testing material for sowing test systems, making the sowing of the testing material on the test system, ensuring the growth of microorganisms with different tolerance to oxygen under specified conditions, controlling the growth of microbes, taking into account the results of this growth and/or the results of the effectiveness of antibiotics, and entering the results into the database and/or sending them to the specified address/destination.

4. The product of claims 1, 3 wherein recording unit of automatic or semiautomatic station/complex that takes into account the results of the growth of microorganisms in the test system and/or the results of the effectiveness of antimicrobials, and adds the results to the database and/or sends them to the specified address/destination.

5. The product of claims 1, 3, 4 wherein monitors the growth and/or takes into account the results of this growth and/or the results of the effectiveness of antibiotics at specified time intervals with a non-limited examples of digital photography, registration in the visible and/or ultraviolet and/or infrared spectra and/or computer-generated imagery with a non-limiting example of calculation the grayscale values and computing histograms, and/or photometry, and/or colorimetry and/or conductivity/resistance of medium of current flow.

6. The product of claims 1, 3-5, wherein test system, in which sensors are integrated, allowing to monitor in various ways the state of individual cells, which changes depending on the presence and nature of the microorganisms' growth and metabolism.

7. The product of claims 1, 3-6, wherein the control of microbial growth on the surface of media is done from an angle of from 1 to 10 and/or 10 to 30 and/or 30-45 and/or 45-90 degrees to the detecting sensor.

8. The product of claims 1, 3-7, wherein the test system contains concentration gradients for each antimicrobial agents in one zone and/or in the form of separate isolated zones, reflecting their pharmacokinetics in certain tissues and organs from zero to the maximum amount.

9. The product of claim 1, wherein Bio adjustable Tetz incubators and/or CO2 incubators and/or bioreactors and/or incubator-warm rooms for the cultivation of bacteria and/or fungi and/or cells, that enable control and/or regulate geomagnetic field and/or electromagnetic exposure and/or alterations of geomagnetic activity and/or alterations of magnetic field of the earth.

10. The product of claims 1, 9, wherein incubators, containers, fermenters, tanks, bioreactors, envelopes, bags, labware, lab supplies, have a shell/part made of Mu metal that provides a given degree of protection and/or their complexes with dielectrics plastic or paper, having a combination of Copper Chambers with zink and plastic or paper.

11. The product of claims 1, 9, 10 wherein incubators, containers, fermenters, tanks, bioreactors, envelopes, bags, labware, lab supplies, culture plates, have a shell/part made of foil and/or their complexes with dielectrics.

12. The product of claims 1, 9-11 wherein incubators, containers, fermenters, tanks, bioreactors, envelopes, bags, labware, lab supplies, culture plates, have a shell/part made of Mu-metal and/or foil and/or their complexes with dielectrics.

13. The product of claims 1, 9-12 wherein incubators, containers, fermenters, tanks, bioreactors, envelopes, bags, labware, lab supplies, culture plates, having a shell/part made of Mu-metal and/or foil and/or their complexes with dielectrics for cultivation of eucaryotic and prokaryotic used for the analysis of the environment, ecology with a non-limiting examples of geomagnetic alterations, magnetic field and/or waves, radiation and health state of the individual from whom cells were collected.

14. The product of claim 1, wherein supplements and additives for nutrient media to regulate growth of different microorganisms including those within mixed microbial communities by the addition of ribavirin (from 0,1-1000 μg/mL), and/or acyclovir (from 0,1-1000 μg/mL), and or lithium orotate from 0,1-1000 ug/mL), and/or potassium orothate (from 0,1-1000 μg/mL), and/or derivatives of 2-chloro-5-phenyl-5H-pyrimido[5′,4′:5,6]pyrano[2,3-d]pyrimidine-4-ol (from 0,1 -1000 μg/mL), and/or nucleases (with a non-limiting examples of DNase (from 0,1-1000 μg/mL) and/or RNase (from 0,1-1000 μg/mL), transcriptase and/or integrase inhibitors and/or protease inhibitors with a non-limiting examples of nevirapine, etravirine, lamivudine, tenofovir, abacavir, raltegravir (from 0,1-1000 μg/mL), that can be used to modulate microbial growth and/or synthetic activity and/or secretion and/or expression of genes, the ratio of growth of different microorganisms including affecting the proportion of Firmicutes, Gracilicutes Mollicutes, acid fast bacteria, yeasts, molds and eukaryotic cells cultures.

15. The product of claim 1, wherein microbial, fungal and eukaryotic cell growth on solid or liquid culture media occurs from 1 minute to 24 hours a day with visible light and/or blue light and/or red light.

16. The product of claim 1, 15 wherein for accelerated growth of fungi (dermatophytes , yeasts, molds) with a non-limiting examples of Candida, Aspergillus, Mucor, Trichophyton, Blastomyces, Cryptococcus, Pneumocystis, Paracoccidioides, Histoplasma, Coccidioides, Talaromyces, Sporothrix. Emmonsia, Fusarium, Malassezia Microsporum Saccharomyces Saprolegnia Erysiphe, Clavicens, Cladosporium. Bipolaris, Shoem, Helmintosporium, Alternaria Penicillium Cladosporium, Alternaria, Epicoccum, Aureobasidium, Absidia Chrysosporium Geotrichum Risopus Eurotium, including combined growth of different fungi and/or bacteria within mixed microbial communities, that can be found in the outer environments, soil, water, books, art objects, objects that interact with humans, animals plants, fungi that cause mammalian, plant diseases comprising of: potato decoction from 0.1 to 500 ml; corn flour tincture from 0.1 to 100 mL; oat flour from 10 to 350 mL; Heart-brain broth from 0,001 to 100,0 g; potato-carrot decoction from 10 to 350 mL; Dextrose/Glucose 40 g; Peptone 10 g; Sucrose from 0,001 to 100,0 g; Cellobiose, from 0,001 to 100,0 g; Yeast extract from 0,001 to 20,0 g; Maltose from 0,001 to 100,0 g; NaNO3 from 0,001 to 10,0 g; K2HPO4 from 0,001 to 10,0 g; MgSO4 from 0,001-10,0 g; KCl from 0,001 to 10,0 g; FeSO4 from 0,001 to 40,0 g; Zn SO4 from 0,001 to 5,0 g; MnCl2 from 0,001 to 5,0 g, Twin 80 from 0,001 to 10 mL, Thiamine from 0,001 to 5,0 mg Biotin from 0,001 to 4,0 mg; heart brain broth 0,001-100,0 g, Agar from 0 to 100 g, orotate derivatives from 0,001 to 500 g; erythrocytes from 0 to 100 mL, antibiotics to inhibit bacterial growth (with a non-limiting examples of chloramphenicol, cephalosporins, tetracyclines) are used.

17. The product of claim 1 15, 16 wherein it is used for the express growth of fungi (dermatophytes , yeasts, molds) with a non-limiting examples of Candida, Aspergillus, Mucor, Trichophyton, Blastomyces, Cryptococcus, Pneumocystis, Paracoccidioides, Histoplasma, Coccidioides, Talaromyces, Sporothrix. Emmonsia, Fusarium, Malassezia Microsporum Saccharomyces Saprolegnia Erysiphe, Clavicens, Cladosporium. Bipolaris, Shoem, Helmintosporium, Alternaria Penicillium Cladosporium, Alternaria, Epicoccum, Aureobasidium, Absidia Chrysosporium Geotrichum Risopus Eurotium.

18. The product of claims 1, 15-17 for the express selection of antibiotics effective against fungi, wherein the antifungal agents are selected from the non-limiting examples of azole derivatives (ketoconazole, fluconazole, isavuconazole, itraconazole, posaconazole, and voriconazole), Echinocandins (anidulafungin, caspofungin, Aminocandin, micafungin), allylamine (terbinafine, Naftin, Tolnaftate), polyene (nystatin, amphotericin B) Flucytosine, Ibrexafungerp, antiseptics, disinfectants.

19. The product of claim 1, wherein for simultaneous growing the maximum number and/or diversity of unrelated microorganisms present at the site of infection, allowing to register the early growth of microorganisms by various methods and to determine the efficiency of the use of antimicrobial agents added to the medium due to their action on Microbial Modulator(s) which control at the site of infection the properties of Primary Pathogen(s), by cultivating biosamples and/or bacteria and/or fungi on the medium: Bile Salt Agar, Thiosulphate Citrate Bile Salts-Sucrose Agar, Bile Esculin Agar, Blood Agar, Chockolate agar, Charcoal Blood Agar, Brain Heart Infusion Broth, Cycloserine Fructose Agar, Cycloserine Egg-Yolk Agar, Egg Saline Medium, Alkaline Egg Medium, Blood-Digest Agar and Broth, Fletcher's Agar, Heated Blood Agar/Chocolate Agar, MacConkey Agar, Mannitol Salt Agar, Hiss's Serum Water Medium, Loeffler Serum Mueller-Hinton Agar, Nutrient Agar, Cooked meat broth, Non-Nutrient Agar Peptone Water, Pike's Media, Anaerobically Sterilized media, Robertson Cooked Meat Broth, Semisolid Agar, Campylobacter Medium, Gram-Negative Broth, Trypticase Soy Broth, Thioglycollate Broth, Trypticase Soy Broth, minimal media, corn meal agar, potato dextrose agar, V-8 juice agar, and dung agar yeast extract, malt extract agar, (Salmonella-Shigella) Agar, Hektoen enteric agar, Listeria comprises a composition of Listeria Broth, DMEM, Fetal Calf Serum, Nalidixic acid, FBS, Tetrathionate Broth, Sabouraud's agar, charcoal yeast extract agar, Mannitol salt agar, LB broth, LB agar, Columbia broth, Columbia agar, Pepted Meat agar, MPB, BcS-LM growth medium, Brucella agar Cornmeal Agar, Water Agar, Emerson's YpSs agar Antibiotic Agar, Acidified Cornmeal Agar, Potato Carrot Agar, Malt Agar, Malt Extract Agar, Potato Dextrose Agar, and a combinations thereof.

20. The product of claim 1 that contains antimicrobial agents added to the medium taken at concentrations (less than maximum concentration at the site of infection) that corresponds to the mean concentration of the antimicrobial agents achievable at the site of infection and/or systemic circulation at different time points after this antimicrobial agents administration with a non-limiting examples of: antimicrobial concentration from 0 to 1 hour (C₀₋₁ₕ), C₁₋₂ₕ, C₀₋₂ₕ, C₁₋₂ₕ, C₀₋₂ₕ, C₁₋₃ₕ, C₂₋₃ₕ, C₀₋₄ₕ, C₁₋₄ₕ, C₂₋₄ₕ, C₃₋₄ₕ, C₀₋₅ₕ, C₁₋₅ₕ, C₂₋₅ₕ, C₃₋₅ₕ, C₄₋₅ₕ, C₀₋₆ₕ, C₁₋₆ₕ, C₂₋₆ₕ, C₃₋₆ₕ, C₄₋₆ₕ, C₅₋₆ₕ, C₀₋₇ₕ, C₁₋₇ₕ, C₂₋₇ₕ, C₃₋₇ₕ, C₄₋₇ₕ, C₅₋₇ₕ, C₀₋₁₂ₕ, C₂₋₁₂ₕ, C₄₋₁₂ₕ, C₄₋₁₂ₕ, C₆₋₁₂ₕ, C₈₋₁₂ₕ, C₀₋₂₄ₕ, C₆₋₂₄ₕ, C₁₂₋₂₄ₕ.

21. The product of claim 1, that contains medium supplemented with antimicrobial agent(s) which concentration is selected from the values that can be reached at the site of infection for the time sufficient to kill and/or inhibit microbial growth (with a non-limiting examples of time within 30 minutes, 2 hours, 3 hours).

22. The product of claims 1 enabling from 2 to 6 h to select antibiotics that are selectively active against certain bacteria and/or fungi within microbial mix, including bacterial-fungal mix.

23. The product of claims 1, 21, 22, wherein antibiotic concentrations added to the system are selected based on particularities of pharmacokinetics that depends on the rout of its administration to the individual with a non-limited examples topically, enterically, orally, parenterally, inhaled, intranasal, rectal, vaginal.

24. The product of claims 1, 21-23, wherein antibiotics are selected to treat bacterial and/or fungal infections with a non-limiting examples of Ear infections, Sinus infections, Cough or bronchitis, Sore throat, pulmonary infections (pneumonia, cystic fibrosis, chronic obstructive pulmonary disease, tuberculosis, mycobacterium, histoplasmosis, blastomycosis bronchiectasis, abscesses, empyema) skin and soft tissue infections (diabetic wound infection, burns, wounds, bites, Impetigo, Cellulitis/Erysipelas, Folliculitis, Skin Abscess, Furuncle, Carbuncle, Necrotizing Soft Tissue Infections), gynecological infections, Maternal infections, ophthalmic infections, oropharyngeal infections, infections of gastrointestinal tract (poisoning, IBD, Inflammatory bowel disease), meningitis, sepsis, fungaemia, systemic mycosis, onychomycosis, urinary tract infections, sexually transmitted diseases, vulvovaginal candidiasis, in individuals with normal or compromised immune response, infections caused by Burkholderia spp, including those in children and patients undergoing lung transplantation, infections associated with persister formation and/or preventing their formation and/or caused by sporeforming microorganisms, and/or recurrent infections.

25. The product of claim 1 wherein culture media and antibiotics and additives are used (taken in different concentrations) that allow the isolation of previously unculturable microorganism (PP and/or MM) from mixed communities.

26. The product of claims 1, 25, wherein antimicrobial effect of drug candidates and/or drugs is evaluated against mixed microbial communities (with a non-limiting examples of microbial communities within biosamples) for (1) comparative analysis with the activity of other drugs (2) select patient population for the clinical trials (3) evaluating the effectiveness of action on humans and animals.

27. The method of claims 2, wherein microbial, fungal and eukaryotic cell growth on solid or liquid culture media occurs from 1 minute to 24 hours a day with visible light and/or blue light and/or red light or infrared light.

28. The method according to claim 2 wherein Mu-metal is used to alter and/or enhance with a non-limiting example microbial growth and/or synthetic activity and/or secretion and/or expression of genes with a non-limiting examples of natural and/or modified and/or engineered eucaryotic or procaryotic producers of molecules and/or proteins of interest, protein expression system, phage display, and those overexpressing recombinant proteins for the use in non-limiting examples of medicine, biotechnology, biomanufacturing, food industry.

29. The method of claim 2 wherein to obtain pure culture of cultivated and not-yet-cultivated microorganisms using a test system in which microorganisms are subculturated from zones and/or wells containing and/or not containing antimicrobial agents.

30. The method of claim 2, wherein the analysis of the signs of appearance and/or progression or absence of the signs of microbial growth (i.e. Primary Pathogen and/or Microorganisms Modulators) is done by visual examination (i.e. naked eye, microscope), or image detection with a non-limiting examples of computer-generated imagery, photography, video, photometry, colorimetry with a non-limiting example of calculation the grayscale values and computing histograms, with or without of automated program and/or AI algorithm and/or (c) inputting the input vectors into a machine learning platform, and/or software; Image Recognition and Image Processing methods, spectrophotometry, scanners, lasers, with a non-limiting examples when the analysis of the surface of the media is done with (i) the certain fixed distance between the sample and the camera (ii) certain wavelength is used, (iii) certain angle between the test system and the camera; is done by comparison of the photo images of the same wells supplemented with antimicrobial agent of interest (with a non-limiting example of time-dependent or concentration dependent) within a certain time period or any combinations thereof and/or comparison with the growth in other wells and/or comparison with a predetermined threshold of growth.

31. The method of claim 2, wherein the analysis of the signs of appearance and/or progression or absence of the signs of microbial growth (i.e. Primary Pathogen and/or Microorganisms Modulators) is done by visual examination (i.e. naked eye, microscope), or image detection with a non-limiting examples of photography, video, computer-generated imagery, photometry, colorimetry with a non-limiting example of calculation the grayscale values and computing histograms, with or without of automated program and/or AI algorithm and/or (c) inputting the input vectors into a machine learning platform, and/or software; Image Recognition and Image Processing methods, spectrophotometry, scanners, lasers, with a non-limiting examples when the analysis of the surface of the media is done with (i) distance between the sample and the camera is from 4 to 5 cm, from 7 to 10 cv, from 12 to 18 cm, from 20-30 cm, from 32.5 to 50 cm (ii) 1×, 1.5×, 2×-10×10×-40×, 40×-1000× magnification (iii) angle between the test system and the camera from 1 to 10 and/or 10 to 30 and/or 30-45 and/or 45-90 degrees angle to the detecting sensor);
is done by comparison of the photo images of the same wells supplemented with antimicrobial agent of interest (with a non-limiting example of time- dependent or concentration dependent) within a certain time period or any combinations thereof and/or comparison with the growth in other wells and/or comparison with a predetermined threshold of growth.

32. The method of claim 2 accounting for results that allow to get data on the effectiveness of antimicrobial agents as soon as possible based on pairwise comparison of microbial growth of the same wells over the different time periods when antibiotic efficacy is evaluated by monitoring of the signs of appearance and/or progression or absence of the signs of microbial growth (i.e. Primary Pathogen and/or Microorganisms Modulators) within a certain time period or any combinations within a non-limiting examples of below listed time periods from: 0 to 1 hour, 0 to 2 h, 1 h to 2 h, 0 h to 3 h, 1 h to 3 h, 2 h to 3 h, 0 h to 4 h, 1 h to 4 h, 2 h to 4 h, 0 h to 5 h, from 1 h to 5 h, 2 h to 5 h, 3 h to 5 h, 0 h to 6 h, 1 h to 6 h, 2 h to 6 h, 3 h to 6 h, 4 h to 6 h, 0 h to 8 h, 1 h to 8 h, 2 h to 8 h, 3 h to 8 h, 4 h to 8 h, 0 h to 9 h, 1 h to 9 h, 2 h to 9 h, 3 h to 9 h, 4 h to 9 h, 5 h to 9 h, 0 h to 12 h, 1 h to 12 h, 2 h to 12 h, 4 h to 12 h, 6 h to 12 h, 8 h to 12 h, 0 h to 18 h, 1 h to 18 h, 2 h to 18 h, 4 h to 18 h, 6 h to 18 h, 8 h to 18 h, 10 h to 18 h, 0 h to 24 h, 1 h to 24 h, 2 h to 24 h, 4 h to 24 h, 12 h to 36 h, 24 h to 36 h.

33. The method of claim 2, wherein antibiotic efficacy is evaluated by monitoring of the signs of appearance and/or progression or absence of the signs of microbial growth (i.e. Primary Pathogen and/or Microorganisms Modulators) on the agar of the wells containing antibiotic of interest at different timepoints including fixed time of 2, 2.5, 3, 3.5, 4, 4.5, 5, 5.5, 6, 6.5, 7, 7.5, 8 hours of growth.

34. The method according to claims 2, 31, 32, wherein antibiotics efficacy models are assigned with different probabilities.

35. The method of claim 2, obtaining pure microbial culture or PP from microbial mix of biosample, wherein the first stage is a combined growth of mixed microorganisms on the media containing the set of antibiotics, with follow up plating of bacteria on different media containing or not containing antibiotics.

36. The product of claim 1 wherein the algorithm of antibiotic selection is based on the evaluation of the highest probability to be effective is determined by which “X” value in “C 1/x max” equation is the highest.
